(19) United States
(12) Patent Application Publication
(10) Pub. No.: US 2026/0284615 A1
(43) Pub. Date: Sep. 24, 2026

(54) FLUIDIC MIXER UNIT DEVICE FOR NANOPARTICLE PRODUCTION

(71) Applicant: AstraZeneca AB, Södertälje (SE)

(72) Inventors: Audrey GALLUD, Södertälje (SE); Alan SABIRSH, Södertälje (SE); Michael UGHETTO, Södertälje (SE)

(21) Appl. No.: 19/472,254

(22) PCT Filed: Apr. 4, 2024
(86) PCT No.: PCT/US2024/022994

§ 371 Date: Oct. 3, 2025

Related U.S. Application Data
(60) Provisional application No. 63/494,776, filed on Apr. 7, 2023.

(51) Int. Cl. B01F 33/301; A61K 9/51; B01F 33/81; B01F 101/22; B33Y 80/00

(52) U.S. Cl. CPC B01F 33/3017; A61K 9/513; A61K 9/5192; B01F 33/813; B33Y 80/00; B01F 2101/22

(57) ABSTRACT

A device 100 for producing nanoparticles includes a microplate 102 which includes a plurality of fluidic mixing units 104 arranged in an array. Each fluidic mixing unit is configured to produce nanoparticles. The device enables high throughput lipid nanoparticle testing and development, and is configured to generate large numbers of novel or unique nanoparticle formulations.

### CROSS-REFERENCE OF RELATED APPLICATIONS

This application claims the benefit of U.S. Provisional Patent Application Ser. No. 63/494,776, filed Apr. 7, 2023, which is hereby incorporated by reference in its entirety for all purposes.

### FIELD

The disclosure relates in general to fluidic mixer devices and methods of nanoparticle production.

### BACKGROUND

Nucleic acids, such as mRNA, have significant potential as a therapeutic drug in many different disease areas as it has the ability to express a protein of interest upon entering a target cell. Although promising as a therapeutic, mRNA faces many challenges from administration to reaching the target cell. Challenges include the limited half-life of mRNA due to exposure to ribonucleases and identification by the immune system, leading to quick degradation and clearance. Finally, the cell membrane of mRNA has a negative potential across the cell surface, creating an electrostatic barrier.

Due to these challenges, mRNA requires protection in order to increase its stability, circulation half-life, cellular delivery and therapeutic effect. Lipid nanoparticles (LNPs) may be utilized for the delivery of mRNAs. Lipid nanoparticles are nanoscale particles composed of a mixture of lipids that are able to enclose or encapsulate a nucleic acid cargo, i.e., mRNA, when an organic phase containing a mixture of lipids and an aqueous phase containing the nucleic acid cargo are appropriately mixed. The encapsulation of mRNA into a lipid nanoparticle is known to counter the challenges that mRNA faces. Lipid nanoparticles can protect mRNA cargoes from degradation and the immune system, thus increasing their half-life, and also facilitate targeting and cellular delivery.

Exploring a large number of lipid components and combinations together with various nucleic acid cargoes assists in identifying efficient delivery vehicles. However, screening studies are currently limited by the commercially available platforms used to formulate lipid nanoparticles, the associated high costs thereof (e.g., single-use microchip, consumables, expensive reagents used in relatively large volumes) and low-throughput approaches. Embodiments hereof relate to devices and methods for nanoparticle production which address these limitations.

### BRIEF SUMMARY

According to a first embodiment hereof, the present disclosure provides a device for producing nanoparticles. The device includes a microplate having a plurality of fluidic mixing units arranged in an array. Each fluidic mixing unit defining a flowpath with each flowpath has a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit. Along the flowpath, the plurality of fluid inlet channels converge downstream into the mixing channel. The array of the plurality of fluidic mixing units includes at least a first set of fluidic mixing units having a first configuration of the flowpath, and a second set of fluidic mixing units having a second configuration of the flowpath. The first configuration is different from the second configuration. Each fluidic mixing unit of the plurality of fluidic mixing units is configured to produce nanoparticles.

In an aspect of the first embodiment, and in combination with any other aspects herein, the disclosure provides that the microplate includes between 50 and 100 fluidic mixing units.

In an aspect of the first embodiment, and in combination with any other aspects herein, the disclosure provides that the plurality of fluid inlet channels of each fluidic mixing unit includes at least three fluid inlet channels.

In an aspect of the first embodiment, and in combination with any other aspects herein, the disclosure provides that during formation of the microplate by 3D printing, the plurality of fluidic mixing units are integrally formed with the microplate.

In an aspect of the first embodiment, and in combination with any other aspects herein, the disclosure provides that each fluidic mixing unit of the plurality of fluidic mixing units includes a first end portion, a second end portion opposing the first end portion and including the single outlet of the fluidic mixing unit, and a waist portion extending between the first end portion and the second end portion. The waist portion has a reduced perimeter relative to the first end portion and the second end portion.

In an aspect of the first embodiment, and in combination with any other aspects herein, the disclosure provides that the plurality of fluidic mixing units further includes a third set of fluidic mixing units having a third configuration of the flowpath. The third configuration is different from each of the first configuration and the second configuration.

In an aspect of the first embodiment, and in combination with any other aspects herein, the disclosure provides that the microplate includes a pattern of inlets formed by the plurality of inlets of each fluidic mixing unit and the pattern of inlets is configured to be compatible with a pipetting robot having a plurality of pipettes.

In an aspect of the first embodiment, and in combination with any other aspects herein, the disclosure provides that the pattern of inlets is a two-dimensional rectangular grid with predetermined spacing between adjacent inlets.

In an aspect of the first embodiment, and in combination with any other aspects herein, the disclosure provides that the predetermined spacing between adjacent inlets is configured to correspond to a spacing of the plurality of pipettes of the pipetting robot.

In an aspect of the first embodiment, and in combination with any other aspects herein, the disclosure provides that the plurality of inlets of each fluidic mixing unit are configured to receive fluid from a pipette of the plurality of pipettes.

In an aspect of the first embodiment, and in combination with any other aspects herein, the disclosure provides that the inlets of the microplate are configured to receive and mate with tips of the plurality of pipettes.

According to a second embodiment hereof, the present disclosure provides a computer-implemented method of manufacturing a device. The method is implemented by at least one processor executing software instructions. The device includes a microplate having a plurality of fluidic mixing units arranged in an array. Each fluidic mixing unit defines a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit. Along the flowpath, the plurality of fluid inlet channels converge downstream into the mixing channel. Each fluidic mixing unit of the plurality of fluidic mixing units is configured to produce nanoparticles. A first fluidic mixing unit object having a first configuration of the flowpath is created by the at least one processor. A second fluidic mixing unit object having a second configuration of the flowpath is created by the at least one processor. The first configuration is different from the second configuration. At least a first set of a plurality of the first fluidic mixing unit objects and a second set of a plurality of the second fluidic mixing unit objects are assembled into an array object by the at least one processor. The device is 3D printed using the array object.

In an aspect of the second embodiment, and in combination with any other aspects herein, the disclosure provides that the step of 3D printing the device includes generating, by the at least one processor, a printing file from the array object and 3D printing the device with the printing file. The printing file has a file format suitable for 3D printing.

In an aspect of the second embodiment, and in combination with any other aspects herein, the disclosure provides that the file format of the printing file is STL.

In an aspect of the second embodiment, and in combination with any other aspects herein, the disclosure provides that the microplate includes between 50 and 100 fluidic mixing units. In an aspect of the second embodiment, and in combination with any other aspects herein, the disclosure provides that each fluidic mixing unit of the plurality of fluidic mixing units includes a first end portion, a second end portion opposing the first end portion and including the single outlet of the fluidic mixing unit, and a waist portion extending between the first end portion and the second end portion. The waist portion has a reduced perimeter relative to the first end portion and the second end portion.

In an aspect of the second embodiment, and in combination with any other aspects herein, the disclosure provides that a third fluidic mixing unit object having a third configuration of the flowpath is created by the at least one processor. The third configuration is different from each of the first configuration and the second configuration.

In an aspect of the second embodiment, and in combination with any other aspects herein, the disclosure provides that the step of assembling includes assembling, by the at least one processor, the first set of the plurality of the first fluidic mixing unit objects, the second set of the plurality of the second fluidic mixing unit objects, and a third set of a plurality of the third fluidic mixing unit objects into the array object.

In an aspect of the second embodiment, and in combination with any other aspects herein, the disclosure provides that the microplate includes a pattern of inlets formed by the plurality of inlets of each fluidic mixing unit and the pattern of inlets is configured to be compatible with a pipetting robot having a plurality of pipettes.

In an aspect of the second embodiment, and in combination with any other aspects herein, the disclosure provides that the pattern of inlets is a two-dimensional rectangular grid with predetermined spacing between adjacent inlets.

In an aspect of the second embodiment, and in combination with any other aspects herein, the disclosure provides that the spacing between adjacent inlets is configured to mate with a spacing of the plurality of pipettes of the pipetting robot.

According to a third embodiment hereof, the present disclosure provides a method for producing lipid nanoparticles with a device. The device includes a microplate having a plurality of fluidic mixing units arranged in an array. Each fluidic mixing unit defines a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit. Along the flowpath, the plurality of fluid inlet channels converge downstream into the mixing channel. Each fluidic mixing unit of the plurality of fluidic mixing units is configured to produce nanoparticles. A lipid solution is injected into a first inlet of the plurality of inlets of each fluidic mixing unit. An mRNA solution is injected into a second inlet and a third inlet of the plurality of inlets of each fluidic mixing unit. The lipid solution and the mRNA solution mix within the mixing channel of each fluidic mixing unit for a time sufficient to form lipid nanoparticles. After mixing, the lipid nanoparticles formed exits the single outlet of each fluidic mixing unit.

In an aspect of the third embodiment, and in combination with any other aspects herein, the disclosure provides that the plurality of fluidic mixing units includes at least a first set of fluidic mixing units having a first configuration of the flowpath and a second set of fluidic mixing units having a second configuration of the flowpath. The first configuration is different from the second configuration.

In an aspect of the third embodiment, and in combination with any other aspects herein, the disclosure provides that the step of injecting the lipid solution into the first inlet of each fluidic mixing unit includes injecting a first lipid solution into the first inlet of a first set of fluidic mixing units and injecting a second lipid solution into the first inlet of a second set of fluidic mixing units. The first lipid solution is different from the second lipid solution.

In an aspect of the third embodiment, and in combination with any other aspects herein, the disclosure provides that the step of injecting the mRNA solution into the second inlet and the third inlet of each fluidic mixing unit includes injecting a first mRNA solution into the second inlet and the third inlet of a first set of fluidic mixing units and injecting a second mRNA solution into the second inlet and the third inlet of a second set of fluidic mixing units. The first mRNA solution is different from the second mRNA solution.

In an aspect of the third embodiment, and in combination with any other aspects herein, the disclosure provides that the lipid solution includes at least one lipid dissolved in a lower alcohol or an organic solvent.

In an aspect of the third embodiment, and in combination with any other aspects herein, the disclosure provides that the at least one lipid includes an ionizable lipid, a cholesterol, a phospholipid, and a PEG lipid.

In an aspect of the third embodiment, and in combination with any other aspects herein, the disclosure provides that the at least one lipid is dissolved in ethanol.

In an aspect of the third embodiment, and in combination with any other aspects herein, the disclosure provides that the mRNA solution includes mRNA dissolved in an aqueous buffer solution.

In an aspect of the third embodiment, and in combination with any other aspects herein, the disclosure provides that the microplate includes between 50 and 100 fluidic mixing units.

In an aspect of the third embodiment, and in combination with any other aspects herein, the disclosure provides that each fluidic mixing unit of the plurality of fluidic mixing units includes a first end portion, a second end portion opposing the first end portion and including the single outlet of the fluidic mixing unit, and a waist portion extending between the first end portion and the second end portion. The waist portion has a reduced perimeter relative to the first end portion and the second end portion.

In an aspect of the third embodiment, and in combination with any other aspects herein, the disclosure provides that the steps of injecting the lipid solution and injecting the mRNA solution are performed simultaneously.

In an aspect of the third embodiment, and in combination with any other aspects herein, the disclosure provides that the steps of injecting the lipid solution and injecting the mRNA solution are performed via a pipetting robot having a plurality of pipettes.

In an aspect of the third embodiment, and in combination with any other aspects herein, the disclosure provides that the microplate includes a pattern of inlets formed by the plurality of inlets of each fluidic mixing unit and the pattern of inlets is configured to be compatible with the pipetting robot.

In an aspect of the third embodiment, and in combination with any other aspects herein, the disclosure provides that the pattern of inlets is a two-dimensional rectangular grid with predetermined spacing between adjacent inlets.

In an aspect of the third embodiment, and in combination with any other aspects herein, the disclosure provides that the spacing between adjacent inlets is configured to mate with a spacing of the plurality of pipettes of the pipetting robot.

According to a fourth embodiment hereof, the present disclosure provides a method of evaluating lipid nanoparticle production with a device. The device includes a microplate having a plurality of fluidic mixing units arranged in an array. Each fluidic mixing unit defines a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit. Along the flowpath, the plurality of fluid inlet channels converge downstream into the mixing channel. The array of the plurality of fluidic mixing units includes at least a first set of fluidic mixing units having a first configuration of the flowpath, and a second set of fluidic mixing units having a second configuration of the flowpath. The first configuration is different from the second configuration. Each fluidic mixing unit of the plurality of fluidic mixing units is configured to produce nanoparticles. A first lipid solution is injected into a first inlet and mRNA into a second inlet and a third inlet of each fluidic mixing unit of the first set of fluidic mixing units. A second lipid solution is injected into a first inlet and mRNA into a second inlet and a third inlet of each fluidic mixing unit of the second set of fluidic mixing units. The first lipid solution and mRNA mix within the mixing channel of each fluidic mixing unit of the first set of fluidic mixing units for a time sufficient to form lipid nanoparticles. After mixing, the lipid nanoparticles formed exits the device through the single outlet of each fluidic mixing unit. The second lipid solution and mRNA mix within the mixing channel of each fluidic mixing unit of the second set of fluidic mixing units for a time sufficient to form lipid nanoparticles. After mixing, the lipid nanoparticles formed exits the device through the single outlet of each fluidic mixing unit. Nanoparticles formed from the first set of fluidic mixing units are evaluated, and nanoparticles formed from the second set of fluidic mixing units are evaluated.

In an aspect of the fourth embodiment, and in combination with any other aspects herein, the disclosure provides that the first lipid solution is the same as the second lipid solution.

In an aspect of the fourth embodiment, and in combination with any other aspects herein, the disclosure provides that the first lipid solution is different from the second lipid solution.

In an aspect of the fourth embodiment, and in combination with any other aspects herein, the disclosure provides that the mRNA is dissolved in an aqueous buffer solution.

In an aspect of the fourth embodiment, and in combination with any other aspects herein, the disclosure provides that each of the first lipid solution and the second lipid solution includes at least one lipid dissolved in a lower alcohol or an organic solvent.

In an aspect of the fourth embodiment, and in combination with any other aspects herein, the disclosure provides that the at least one lipid is selected from the group consisting of an ionizable lipid, a cholesterol, a phospholipid, and a PEG lipid.

In an aspect of the fourth embodiment, and in combination with any other aspects herein, the disclosure provides that the at least one lipid includes an ionizable lipid, a cholesterol, a phospholipid, and a PEG lipid.

In an aspect of the fourth embodiment, and in combination with any other aspects herein, the disclosure provides that the at least one lipid is dissolved in ethanol.

In an aspect of the fourth embodiment, and in combination with any other aspects herein, the disclosure provides that the microplate includes between 50 and 100 fluidic mixing units.

In an aspect of the fourth embodiment, and in combination with any other aspects herein, the disclosure provides that each fluidic mixing unit of the plurality of fluidic mixing units includes a first end portion, a second end portion opposing the first end portion and including the single outlet of the fluidic mixing unit, and a waist portion extending between the first end portion and the second end portion. The waist portion has a reduced perimeter relative to the first end portion and the second end portion.

In an aspect of the fourth embodiment, and in combination with any other aspects herein, the disclosure provides that the steps of injecting are performed simultaneously.

In an aspect of the fourth embodiment, and in combination with any other aspects herein, the disclosure provides that the steps of injecting are performed via a pipetting robot having a plurality of pipettes.

In an aspect of the fourth embodiment, and in combination with any other aspects herein, the disclosure provides that the microplate includes a pattern of inlets formed by the plurality of inlets of each fluidic mixing unit and the pattern of inlets is configured to be compatible with the pipetting robot.

In an aspect of the fourth embodiment, and in combination with any other aspects herein, the disclosure provides that the pattern of inlets is a two-dimensional rectangular grid with predetermined spacing between adjacent inlets.

In an aspect of the fourth embodiment, and in combination with any other aspects herein, the disclosure provides that the spacing between adjacent inlets is configured to mate with a spacing of the plurality of pipettes of the pipetting robot.

In an aspect of the fourth embodiment, and in combination with any other aspects herein, the disclosure provides that the nanoparticles formed from the first configuration are compared with the nanoparticles formed from the second configuration.

In an aspect of the fourth embodiment, and in combination with any other aspects herein, the disclosure provides that the step of evaluating the nanoparticles formed from the first set of fluidic mixing units and the step of evaluating the nanoparticles formed from the second set of fluidic mixing units include evaluating at least one parameter of the nanoparticles, the parameter being selected from the group consisting of a size of the nanoparticles, a surface charge of the nanoparticles and an encapsulation efficiency of the nanoparticles.

According to a fifth embodiment hereof, the present disclosure provides a method of evaluating lipid nanoparticle production with a device. The device includes a microplate having a plurality of fluidic mixing units arranged in an array. Each fluidic mixing unit defines a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit. Along the flowpath, the plurality of fluid inlet channels converge downstream into the mixing channel. The array of the plurality of fluidic mixing units includes at least a first set of fluidic mixing units and a second set of fluidic mixing units. Each fluidic mixing unit of the plurality of fluidic mixing units is configured to produce nanoparticles. A first lipid solution is injected into a first inlet and mRNA into a second inlet and a third inlet of each fluidic mixing unit of the first set of fluidic mixing units. A second lipid solution is injected into a first inlet and mRNA into a second inlet and a third inlet of each fluidic mixing unit of the second set of fluidic mixing units. The first lipid solution is different from the second lipid solution. The first lipid solution and the mRNA mix within the mixing channel of each fluidic mixing unit of the first set of fluidic mixing units for a time sufficient to form lipid nanoparticles. After mixing, the lipid nanoparticles formed exits the device through the single outlet of each fluidic mixing unit. The second lipid solution and the mRNA mix within the mixing channel of each fluidic mixing unit of the second set of fluidic mixing units for a time sufficient to form lipid nanoparticles. After mixing, the lipid nanoparticles formed exits the device through the single outlet of each fluidic mixing unit. The nanoparticles formed from the first set of fluidic mixing units are evaluated, and the nanoparticles formed from the second set of fluidic mixing units are evaluated.

In an aspect of the fifth embodiment, and in combination with any other aspects herein, the disclosure provides that the first set of fluidic mixing units has a first configuration of the flowpath, and the second set of fluidic mixing units has a second configuration of the flowpath. The first configuration is different from the second configuration.

In an aspect of the fifth embodiment, and in combination with any other aspects herein, the disclosure provides that the first set of fluidic mixing units has a first configuration of the flowpath, and the second set of fluidic mixing units has a second configuration of the flowpath. The first configuration is the same as the second configuration.

In an aspect of the fifth embodiment, and in combination with any other aspects herein, the disclosure provides that the mRNA is dissolved in an aqueous buffer solution.

In an aspect of the fifth embodiment, and in combination with any other aspects herein, the disclosure provides that each of the first lipid solution and the second lipid solution includes at least one lipid dissolved in a lower alcohol or an organic solvent.

In an aspect of the fifth embodiment, and in combination with any other aspects herein, the disclosure provides that the at least one lipid is selected from the group consisting of an ionizable lipid, a cholesterol, a phospholipid, and a PEG lipid.

In an aspect of the fifth embodiment, and in combination with any other aspects herein, the disclosure provides that the at least one lipid is selected from the group consisting of an ionizable lipid, a cholesterol, a phospholipid, and a PEG lipid.

In an aspect of the fifth embodiment, and in combination with any other aspects herein, the disclosure provides that the at least one lipid includes an ionizable lipid, a cholesterol, a phospholipid, and a PEG lipid.

In an aspect of the fifth embodiment, and in combination with any other aspects herein, the disclosure provides that the at least one lipid is dissolved in ethanol.

In an aspect of the fifth embodiment, and in combination with any other aspects herein, the disclosure provides that the microplate includes between 50 and 100 fluidic mixing units.

In an aspect of the fifth embodiment, and in combination with any other aspects herein, the disclosure provides that each fluidic mixing unit of the plurality of fluidic mixing units includes a first end portion, a second end portion opposing the first end portion and including the single outlet of the fluidic mixing unit, and a waist portion extending between the first end portion and the second end portion. The waist portion has a reduced perimeter relative to the first end portion and the second end portion.

In an aspect of the fifth embodiment, and in combination with any other aspects herein, the disclosure provides that the steps of injecting are performed simultaneously.

In an aspect of the fifth embodiment, and in combination with any other aspects herein, the disclosure provides that the steps of injecting are performed via a pipetting robot having a plurality of pipettes.

In an aspect of the fifth embodiment, and in combination with any other aspects herein, the disclosure provides that the microplate includes a pattern of inlets formed by the plurality of inlets of each fluidic mixing unit and the pattern of inlets is configured to be compatible with the pipetting robot.

In an aspect of the fifth embodiment, and in combination with any other aspects herein, the disclosure provides that the pattern of inlets is a two-dimensional rectangular grid with predetermined spacing between adjacent inlets.

In an aspect of the fifth embodiment, and in combination with any other aspects herein, the disclosure provides that the spacing between adjacent inlets is configured to mate with a spacing of the plurality of pipettes of the pipetting robot.

In an aspect of the fifth embodiment, and in combination with any other aspects herein, the disclosure provides that the nanoparticles formed from the first configuration are compared with the nanoparticles formed from the second configuration.

In an aspect of the fifth embodiment, and in combination with any other aspects herein, the disclosure provides that the step of evaluating the nanoparticles formed from the first set of fluidic mixing units and the step of evaluating the nanoparticles formed from the second set of fluidic mixing units include evaluating at least one parameter of the nanoparticles. The parameter is selected from the group consisting of a size of the nanoparticles, a surface charge of the nanoparticles and an encapsulation efficiency of the nanoparticles.

### BRIEF DESCRIPTION OF DRAWINGS

The foregoing and other features and advantages of the disclosure will be apparent from the following description of embodiments hereof as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 is a perspective view of a device according to an embodiment hereof, the device including a microplate having a plurality of fluidic mixing units arranged in an array.

FIG. 2 is a perspective view of an embodiment of a fluidic mixing unit of the device of FIG. 1.

FIG. 3 is a semi-transparent perspective view of the fluidic mixing unit of FIG. 2, illustrating a flowpath thereof.

FIG. 3A is a schematic side view of the fluidic mixing unit of FIG. 2 having the flowpath separated and external therefrom for illustrative purposes only.

FIG. 4 is a flow chart of a method of producing lipid nanoparticles with the fluidic mixing unit of FIG. 2.

FIG. 5 is a schematic illustration of a lipid solution and mRNA being injected into the fluidic mixing unit of FIG. 2.

FIG. 6A is a schematic illustration of various arrays including the fluidic mixing unit of FIG. 2.

FIG. 6B is another perspective view of the device of FIG. 1.

FIG. 6C is an enlarged perspective view of a portion of a top surface of the device of FIG. 1.

FIG. 6D is an enlarged perspective view of a portion of a bottom surface of the device of FIG. 1.

FIG. 7 is an enlarged perspective view of a portion of a top surface of a well plate that may be used with the device of FIG. 1.

FIG. 7A is a perspective view of an alternative well plate that may be used with the device of FIG. 1.

FIG. 8 is a perspective view of a device according to an embodiment hereof, the device including a microplate having a plurality of fluidic mixing units arranged in an array, wherein the array includes a first set of fluidic mixing units according to an embodiment and a second set of fluidic mixing units according to an embodiment.

FIG. 8A is a flow chart of a method of producing lipid nanoparticles with the device of FIG. 8 according to an embodiment hereof.

FIG. 9 is a semi-transparent perspective view of a fluidic mixing unit according to another embodiment hereof, illustrating a flowpath thereof.

FIG. 10 is a semi-transparent perspective view of a fluidic mixing unit according to another embodiment hereof, with an illustrating a flowpath thereof.

FIG. 11 is a semi-transparent perspective view of a fluidic mixing unit according to another embodiment hereof, illustrating a flowpath thereof.

FIG. 12 is a semi-transparent perspective view of a fluidic mixing unit according to another embodiment hereof, illustrating a flowpath thereof.

FIG. 13A illustrates a step of a method of using the device of FIG. 1 with a pipetting robot and an automated platform according to an embodiment hereof.

FIG. 13B illustrates a step of the method of using the device of FIG. 1 with the pipetting robot and the automated platform of FIG. 13A.

FIG. 13C illustrates a step of the method of using the device of FIG. 1 with the pipetting robot and the automated platform of FIG. 13A.

FIG. 13D illustrates a step of the method of using the device of FIG. 1 with the pipetting robot and the automated platform of FIG. 13A.

FIG. 13E illustrates a step of the method of using the device of FIG. 1 with the pipetting robot and the automated platform of FIG. 13A.

FIG. 13F illustrates a step of the method of using the device of FIG. 1 with the pipetting robot and the automated platform of FIG. 13A.

FIG. 13G illustrates a step of the method of using the device of FIG. 1 with the pipetting robot and the automated platform of FIG. 13A.

FIG. 13H illustrates a step of the method of using the device of FIG. 1 with the pipetting robot and the automated platform of FIG. 13A.

FIG. 13I illustrates a step of the method of using the device of FIG. 1 with the pipetting robot and the automated platform of FIG. 13A.

FIG. 14 is a flow chart of a computer-implemented method of manufacturing a device according to an embodiment hereof, the device including a microplate having a plurality of fluidic mixing units arranged in an array.

FIG. 15A is a chart illustrating a diameter or size of benchmark lipid nanoparticles formulated on the NanoAssemblr® GMP system compared to a diameter or size of lipid nanoparticles prepared by the devices and/or methods described herein.

FIG. 15B is a chart illustrating a polydispersity (PD) index of benchmark lipid nanoparticles formulated on the NanoAssemblr® GMP system compared to a polydispersity (PD) index of lipid nanoparticles prepared by the devices and/or methods described herein.

FIG. 15C is a chart illustrating an encapsulation percentage of benchmark lipid nanoparticles formulated on the NanoAssemblr® GMP system compared to an encapsulation percentage of lipid nanoparticles prepared by the devices and/or methods described herein.

FIG. 15D is a chart illustrating a total polyA concentration of benchmark lipid nanoparticles formulated on the NanoAssemblr® GMP system compared to a total polyA concentration of lipid nanoparticles prepared by the devices and/or methods described herein.

### DETAILED DESCRIPTION

Specific embodiments of the present disclosure are now described with reference to the figures, wherein like reference numbers indicate identical or functionally similar elements. The following detailed description is merely exemplary in nature and is not intended to limit the disclosure or the application and uses of the disclosure. Although the description of the disclosure is in the context of lipid nanoparticles production, the disclosure may also be used to produce other materials where it is deemed useful. Furthermore, there is no intention to be bound by any expressed or implied theory presented in the preceding technical field, background, brief summary or the following detailed description.

Embodiments hereof relate to a device having a plurality of fluidic mixing units arranged in an array, allowing the miniaturization of lipid nanoparticle formulations while maintaining reproducibility and particle profiles similar to industrial standards. With equivalent process duration and cost range, a plurality of lipid nanoparticles can be simultaneously formulated at scales twenty to fifty times smaller than a single standard formulation using known low-throughput solutions. The device enables testing and development of unique or novel lipid nanoparticles in a systematic and high throughput manner and may be used in screening studies which aim to identify active nanoparticles for delivery of mRNA therapeutics. Screening studies are used to evaluate lipid nanoparticle attributes, thus producing lipid nanoparticles which meet industry standards in terms of quality but with much higher throughput and significantly less cost as compared to known low-throughput solutions. Stated another way, different lipid solutions are screened or evaluated to determine the effect on lipid nanoparticle attributes, such as diameter size, polydispersity index, surface charge, encapsulation efficiency and mRNA concentration. New assays can be added over time, and the experimental infrastructure allows more project-specific questions (cell models, targeting ligands, cargo designs) in various contexts. Data from the screening studies may be utilized for data modelling approaches and may drive new lipid chemistry. Modelling can highlight inactive lipids and guide lipid selections when moving novel lipids to in-vivo studies.

In addition to enabling simultaneous screening of different lipid solutions, the device also enables screening of geometry variations of the fluidic mixing units to determine an optimal geometry. Different geometries of the fluidic mixing units may be tested on the same device. As such, the physical design or geometry of the fluidic mixing units is an experimental parameter which can be optimized for particular delivery systems and applications in a way that is not currently possible with known micro-mixing systems for nanoparticle production.

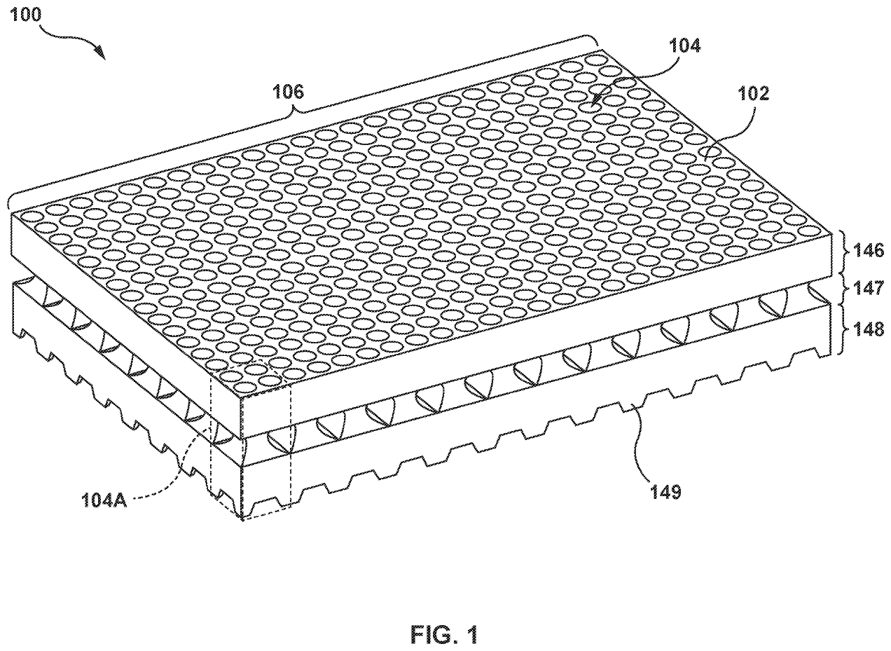

Turning now to the figures, a device for producing nanoparticles will be described in more detail. FIG. 1 illustrates a perspective view of a device 100 according to an embodiment hereof. The device 100 includes a unitary or one-piece microplate 102 which includes a plurality of fluidic mixing units 104 integrated or arranged in an array 106. As will be described in more detail herein, each fluidic mixing unit 104 is configured to produce nanoparticles independently of adjacent or neighboring mixing units. In the depicted embodiment, the device 100 is configured to make ninety-six (96) different types of nanoparticles simultaneously; thus the device 100 enables high throughput lipid nanoparticle testing and development. The device 100 is configured to generate large numbers of novel or unique nanoparticle variations at many times the speed of conventional devices, while also reducing the cost compared to competing formulation methodologies. Further, the device 100 is reusable.

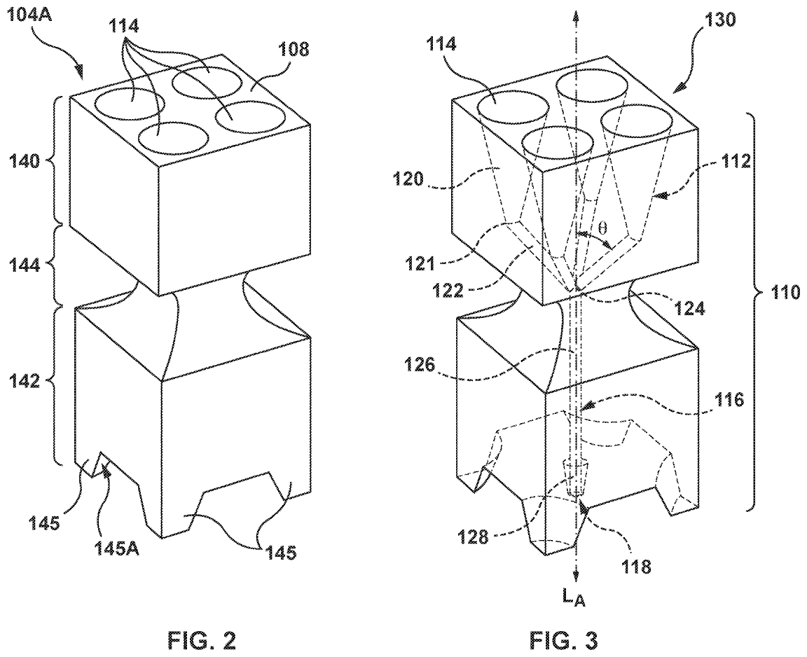

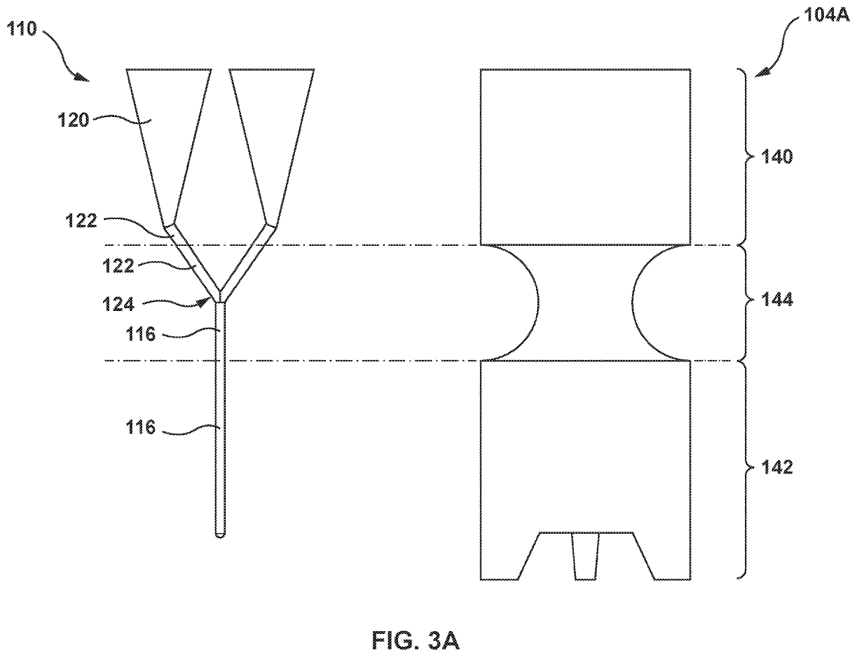

With reference to FIGS. 2 and 3, each fluidic mixing unit 104 of the plurality of fluidic mixing units 104 includes a body 108 that defines an internal geometry or flowpath 110, wherein the flowpath 110 extends from a first or top surface to a second or bottom surface of the microplate 102. Each flowpath 110 has a plurality of fluid inlet channels 112. Each fluid inlet channel 112 includes an inlet 114 such that, collectively, the inlets 114 of the plurality of fluid inlet channels 112 define a plurality of inlets of the respective fluidic mixing unit 104. Each flowpath 110 also includes a mixing channel 116. The mixing channel 116 includes an outlet 118 that defines a single outlet of the respective fluidic mixing unit 104. Along the flowpath 110, at a junction 124, the plurality of fluid inlet channels 112 converge downstream into the mixing channel 116. As used herein, the reference number 104 refers to a fluidic mixing unit having a flowpath, but the flowpath does not have a specified or particular configuration. Stated another way, when reference number 104 is utilized herein with reference to a fluidic mixing unit, the corresponding description is general and applies to all fluidic mixing units of the array 106, regardless of the geometry or design of the flowpath.

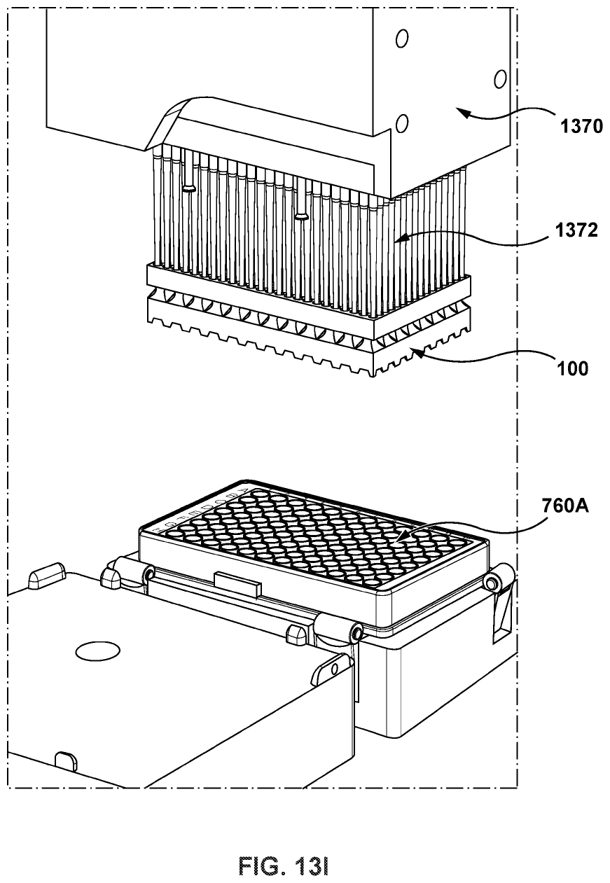

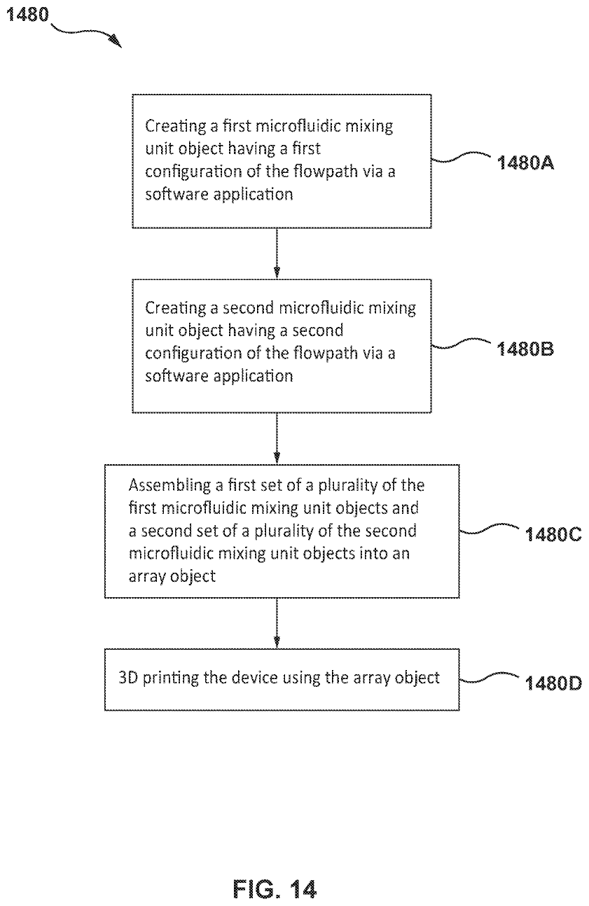

In an embodiment, each fluidic mixing unit 104 of the plurality of fluidic mixing units 104 is a large microfluidic mixing unit. As used herein, “large microfluidic” includes devices or units having channels having a width dimension between 100 μm to 5000 μm, as opposed to traditional microfluidic devices which includes devices having channels of a width dimension less than 100 μm. In an embodiment, the mixing channel 116 of each fluidic mixing unit 104 has a width between 100 μm and 1000 μm. In an embodiment, the mixing channel 116 of each fluidic mixing unit 104 has a width between 200 μm and 900 μm. In an embodiment, the mixing channel 116 of each fluidic mixing unit 104 has a width between 300 μm and 700 μm. In an embodiment, the mixing channel 116 of each fluidic mixing unit 104 has a width between 400 μm and 600 μm. The dimensions described above are the actual dimensions in the device 100 as opposed to the theoretical dimensions which are input into a processor as described herein with respect to FIG. 14. The actual dimensions may be the same as the theoretical dimensions, or the actual dimensions may be slightly larger or slightly smaller than the theoretical dimensions.

In an embodiment, each of the fluid inlet channel 112 and the mixing channel 116 has a circular cross-section along an entire length thereof. However, embodiments hereof are not limited to channels having a particular cross-section. For example, depending upon the application or intended use of the device 100, one or more of the fluid inlet channels 112 and the mixing channel 116 may have an elliptical or polygonal cross-section. Further, depending upon the application or intended use of the device 100, each fluid inlet channel 112 and the mixing channel 116 of each fluidic mixing unit 104 need not have a consistent cross-section along the length thereof. In an embodiment, the cross-sectional shape of one or more of the fluid inlet channels 112 and the mixing channel 116 may vary along a length thereof.

FIG. 2 is a perspective view of a fluidic mixing unit according to an embodiment hereof, which is also labeled and referred to herein as fluidic mixing unit 104A. As shown in FIG. 1, the fluidic mixing unit 104A is integrally and seamlessly formed with the remainder of the microplate 102 and thus the fluidic mixing unit 104A is shown isolated or removed from the remainder of the microplate 102 in FIG. 2 for description purposes only. FIG. 3 is a semi-transparent perspective view of the fluidic mixing unit 104A showing a flowpath 110 thereof. The flowpath 110 extends between the top and bottom surfaces of the microplate 102. The fluidic mixing unit 104A has a first configuration or geometry 130 of the flowpath 110. The first configuration 130 includes four fluid inlet channels 112. In an embodiment, each of the four fluid inlet channels 112 have the same shape and size. Each fluid inlet channel 112 includes a generally cone-shaped portion 120 and a cylindrical portion 122. The generally cone-shaped portion 120 extends between the inlet 114 and the cylindrical portion 122, and the cylindrical portion 122 extends between the generally cone-shaped portion 120 and the junction 124. As used herein, “generally cone-shaped” includes cylinders having different diameters at opposing ends thereof. More particularly, a diameter of the generally cone-shaped portion 120 is variable and decreases/tapers in a direction towards the cylindrical portion 122, with the inlet 114 having a larger diameter than an opposing end 121 of the generally cone-shaped portion 120. In an embodiment, a diameter of the inlet 114 may be up to ten (10) times greater than the opposing or downstream end of the generally cone-shaped portion 120. In an embodiment, the diameter of the inlet 114 is between 3000 μm and 4000 μm, while the opposing end 121 of the cone-shaped portion 120 is between 300 μm and 800 μm The generally cone-shaped portion 120 extends parallel or substantially parallel to a longitudinal axis L_{A} of the fluidic mixing unit 104A.

Notably, as will be described in more detail herein, each generally cone-shaped portion 120 of the fluid inlet channels 112 of the microplate 102 is configured to receive and mate with a pipette tip of a pipetting robot. The shape and size of the generally cone-shaped portion 120 is designed and optimized to ensure engagement between the pipette tip (not shown) and the generally cone-shaped portion 120 of the fluid inlet channels 112. More particularly, each generally cone-shaped portion 120 of the fluid inlet channels 112 is configured such that a pipette tip extends a sufficient amount into the generally cone-shaped portion 120 to create an adequate seal therewith and thereby prevent leakage, but does not extend so far into the generally cone-shaped portion 120 such that the pipette tip is pinched and possibly blocking the fluid inlet channel 112.

The cylindrical portion 122 of each fluid inlet channel 112 has a constant diameter along a length thereof, which may range between 300 μm and 800 μm, and is angled from its corresponding generally cone-shaped portion 120 toward the longitudinal axis L_{A} of the fluidic mixing unit 104A. In an embodiment, the diameter of the cylindrical portion 122 is equal to or substantially equal to the diameter of the downstream end of the generally cone-shaped portion 120. The cylindrical portion 122 extends at an angle θ relative to the longitudinal axis L_{A} of the fluidic mixing unit 104A. In an embodiment, the angle θ ranges between 30-60 degrees.

At the junction 124, the four fluid inlet channels 112 converge or transition into the mixing channel 116. In the first configuration 130 of the flowpath 110, the mixing channel 116 has a cylindrical portion 126 and an exit or tip portion 128. The cylindrical portion 126 extends between the junction 124 and the tip portion 128, and the tip portion 128 extends between the cylindrical portion 126 and the outlet 118 of the mixing channel 116. The cylindrical portion 126 extends parallel or substantially parallel to the longitudinal axis L_{A} of the fluidic mixing unit 104. The cylindrical portion 126 has a constant diameter along a length thereof, which may range between 300 μm and 800 μm. The tip portion 128 also extends parallel or substantially parallel to the longitudinal axis L_{A} of the fluidic mixing unit 104. In an embodiment, the tip portion 128 has a diameter which is greater than the diameter of the cylindrical portion 126. The diameter of the tip portion 128 may range between 300 μm and 1000 μm, and may be constant along a length thereof or may be varied.

In order to avoid unintentional damage to or breakage of the tip portion 128, an outer surface of the fluidic mixing unit 104A along the tip portion 128 may have a frustoconical configuration. More particularly, a thickness of the wall that forms the tip portion 128 may decrease from an inflow end to an outflow end thereof. The increased wall thickness at the inflow end of the tip portion 128 reinforces the structure forming the tip portion 128 to strengthen the tip portion 128 during use of the microplate 102. In an embodiment, an outer diameter of the structure forming the tip portion 128 may be between 600 μm and 1000 μm at the inflow end of the tip portion 128 and an outer diameter of the structure forming the tip portion 128 may be between 90 μm and 250 μm at the outflow end of the tip portion 128. The decrease in outer diameter along the structure forming the tip portion 128 is due to a gradual or tapered decrease in wall thickness along the length of the tip portion 128 as described above.

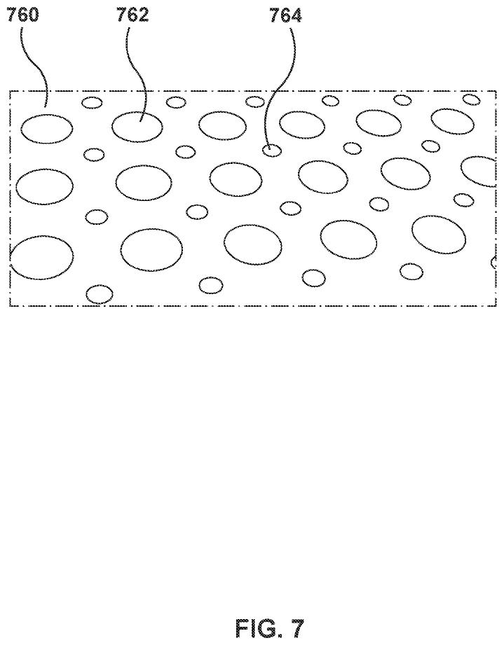

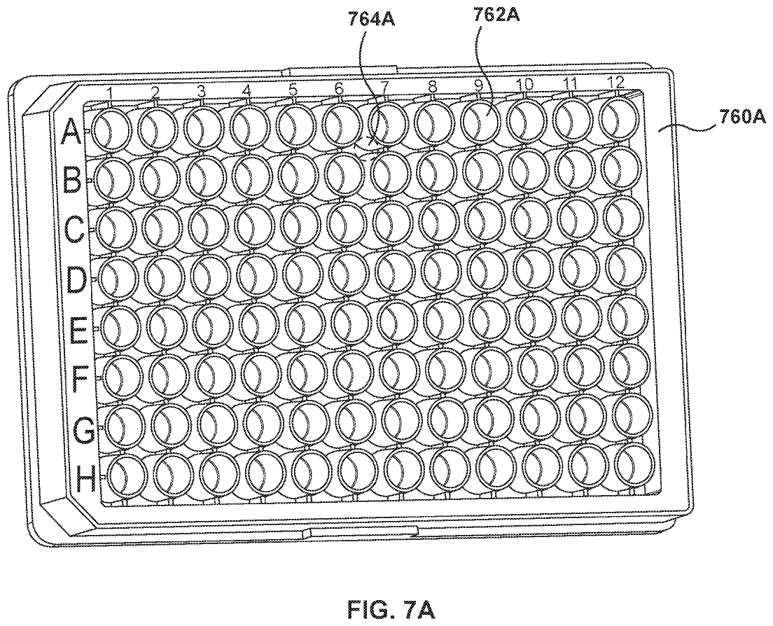

With additional reference to FIG. 3A, which is a schematic side view of the fluidic mixing unit 104A having the flowpath 110 separated and external therefrom for illustrative purposes only, the fluidic mixing unit 104A may be considered to include a first or inflow end portion 140, a second or outflow end portion 142 opposing the first end portion 140 and a waist portion 144 extending between the first end portion 140 and the second end portion 142. The first end portion 140 includes the four inlets 114 of the fluidic mixing unit 104A, the generally cone-shaped portions 120 of the fluid inlet channels 112, and the top portions of the cylindrical portions 122 of the fluid inlet channels 112. The waist portion 144 includes the remaining lengths of the cylindrical portions 122 of the fluid inlet channels 112, the junction 124, and a top portion of the mixing channel 116. The waist portion 144 of the fluidic mixing unit 104A is formed by four curved surfaces that extend radially inward such that the waist portion 144 has a reduced perimeter relative to the first end portion 140 and the second end portion 142. The second end portion 142 includes the remaining length of the mixing channel 116 and the single outlet 118 of the fluidic mixing unit 104A. The second end portion 142 also includes a plurality of corner posts 145. Each corner post 145 has an inwardly-facing surface 145A that is rounded or arc-shaped. As further described herein with respect to FIG. 7, the corner posts 145 function to recess the bottom surface of the fluidic mixing unit such that the microplate 102 is configured to mate with a well plate that collects the formed lipid nanoparticles.

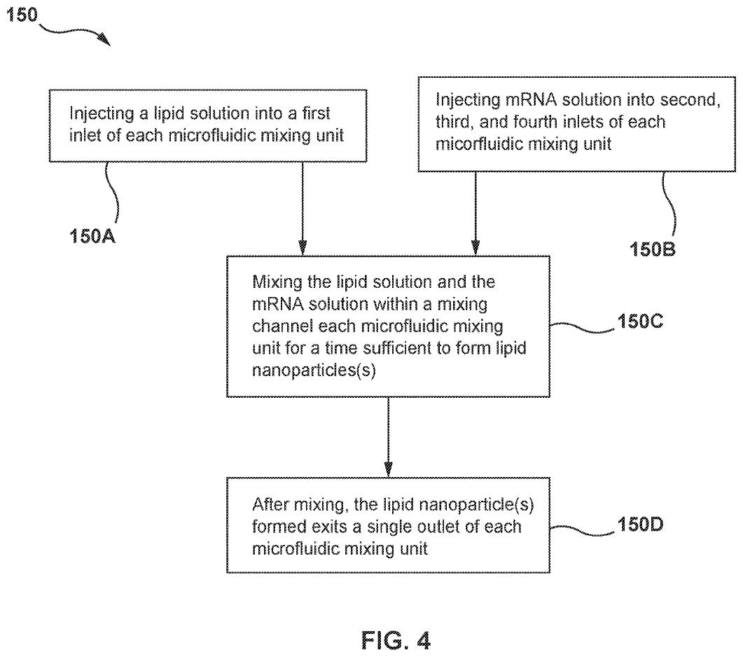

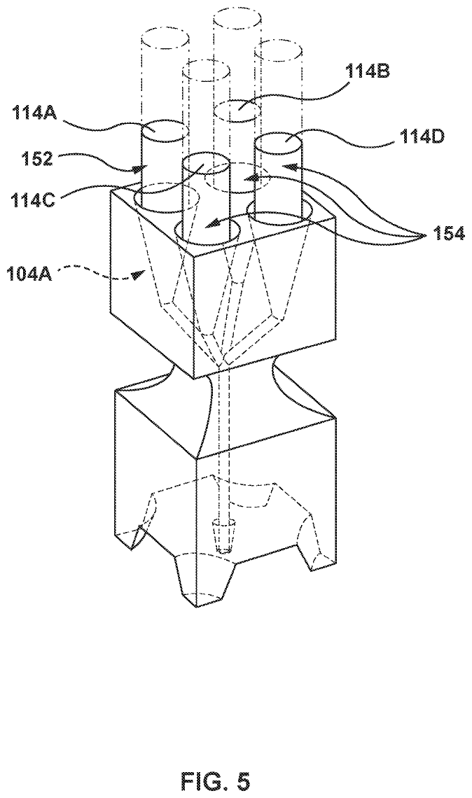

Each fluidic mixing unit 104A is configured to produce one type or formulation of lipid nanoparticles. With reference to FIGS. 4 and 5, a method 150 of producing lipid nanoparticles within the fluidic mixing unit 104A is described in more detail. FIG. 4 depicts a flow chart of a method for producing lipid nanoparticles with the fluidic mixing unit 104A, while FIG. 5 is a schematic illustration of material components for producing lipid nanoparticles being injected into the fluidic mixing unit 104A.

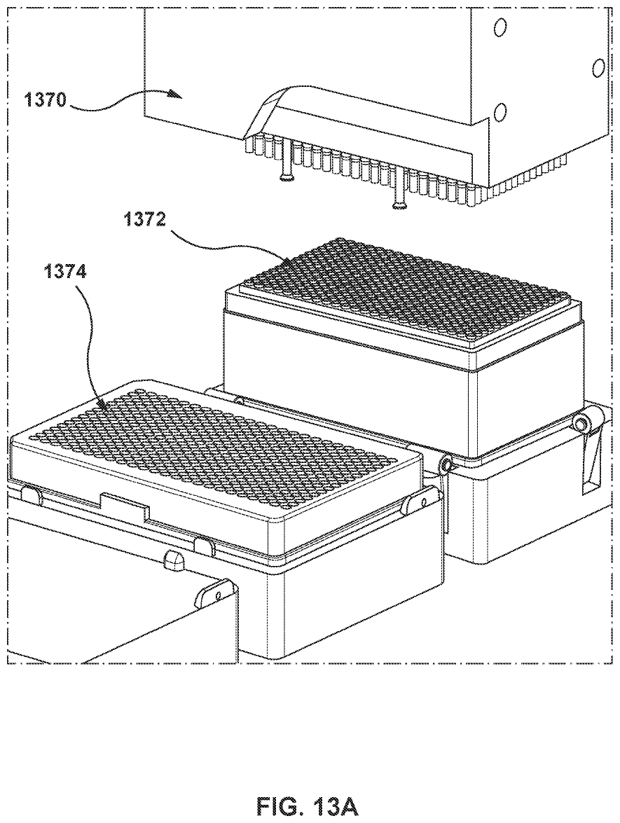

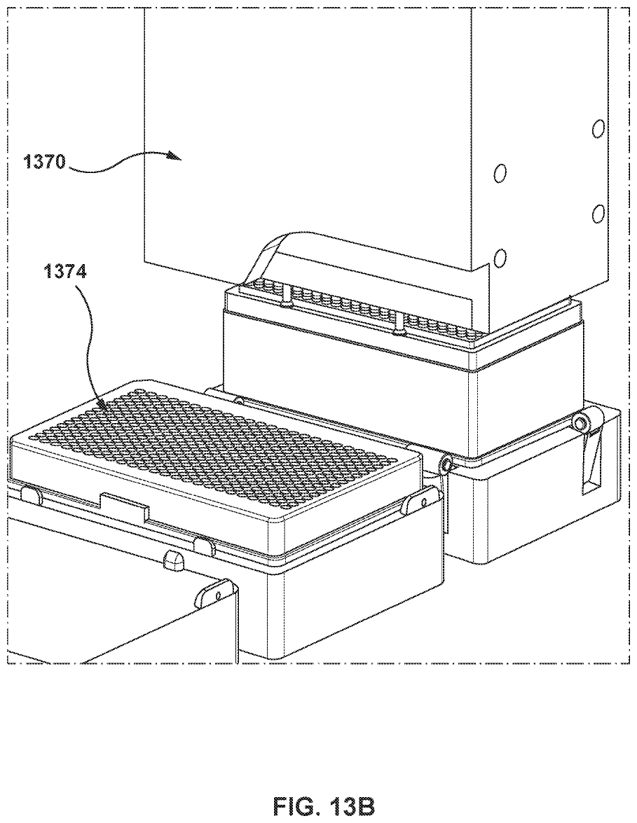

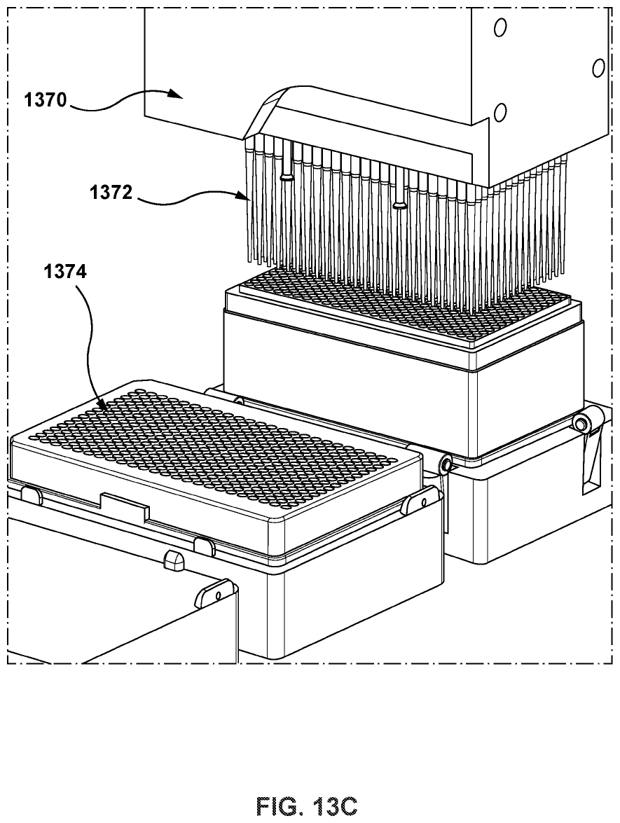

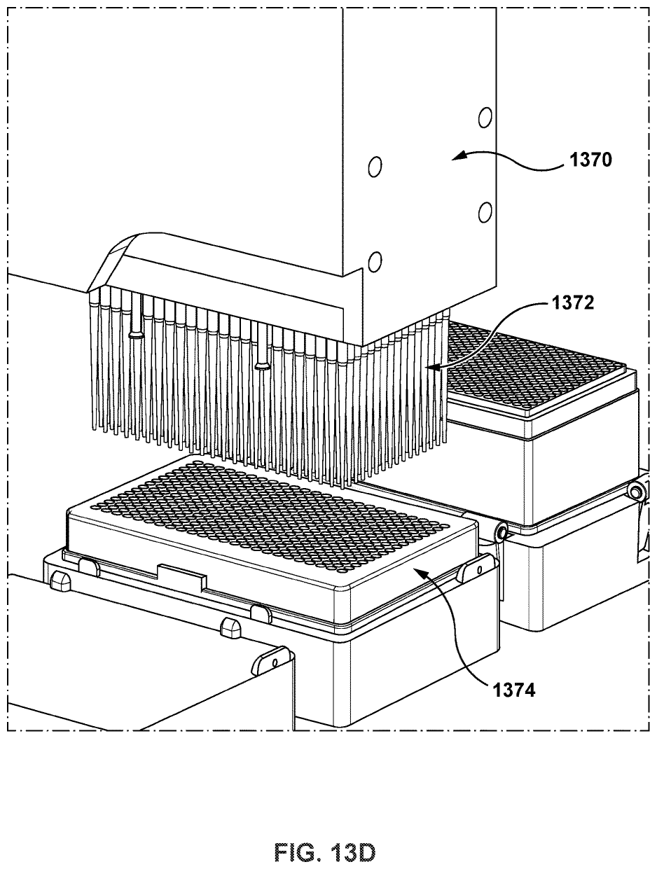

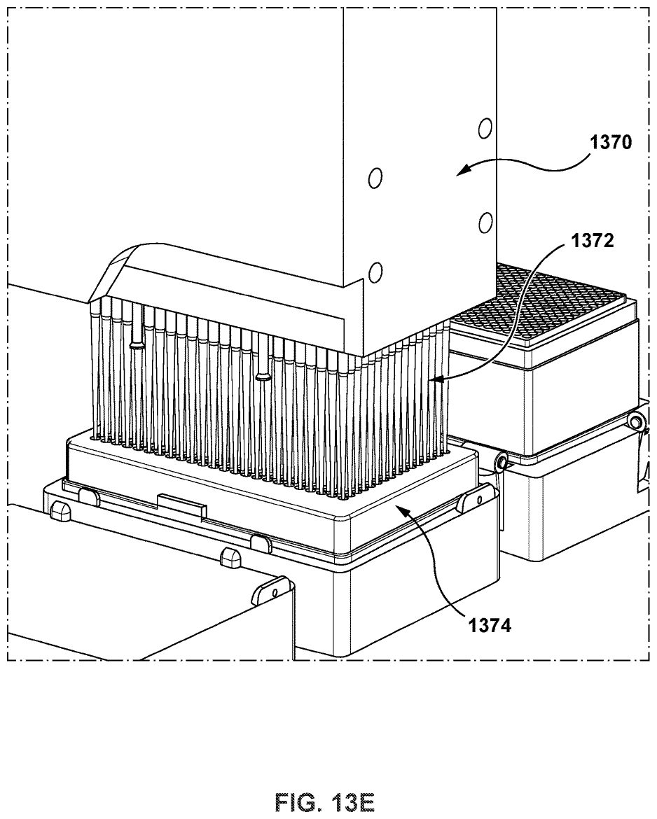

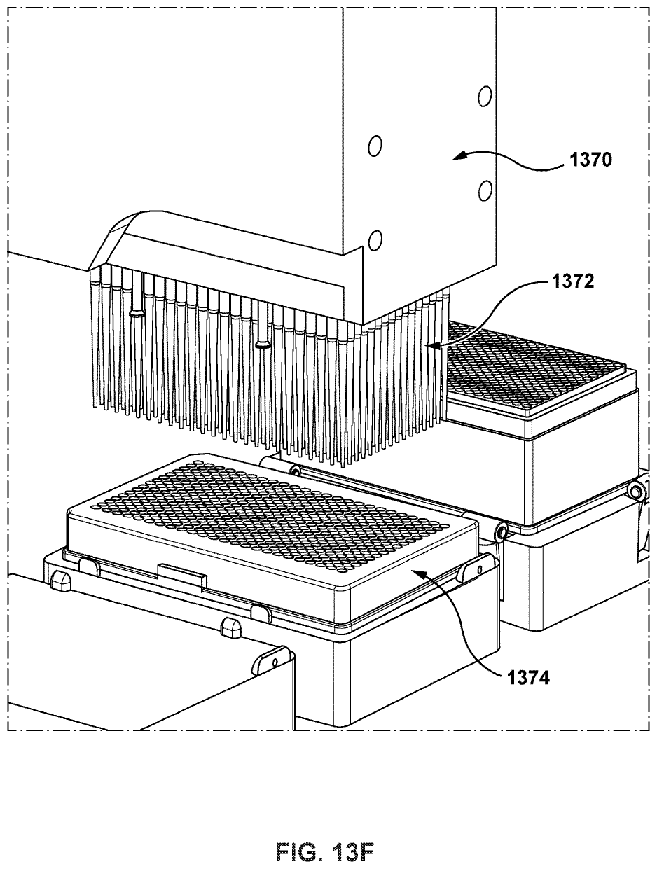

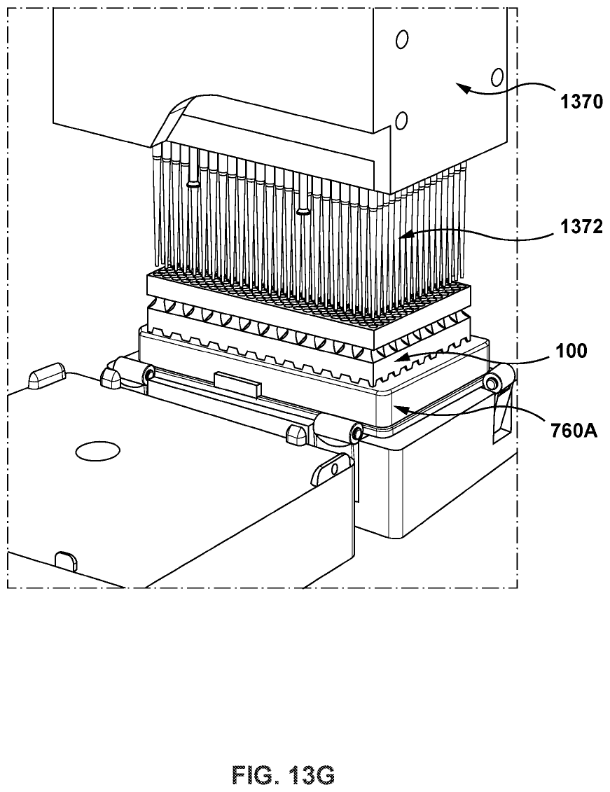

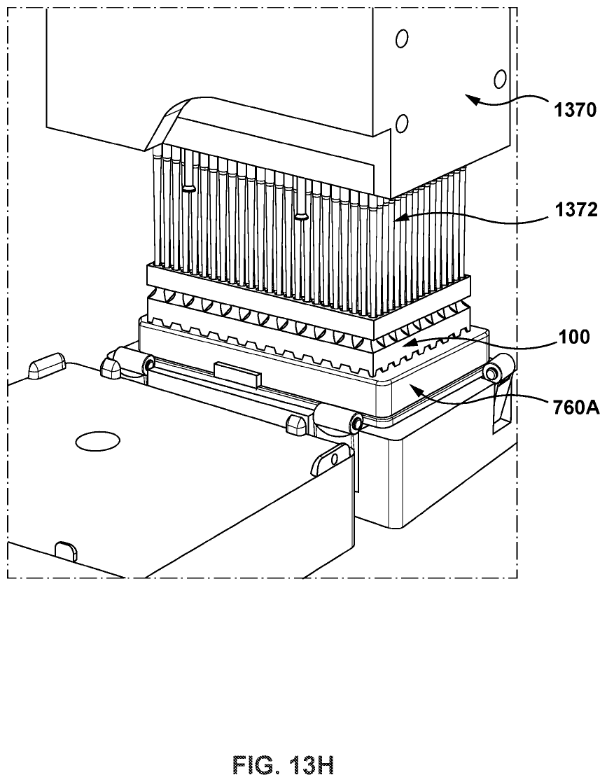

With reference to steps 150A and 150B of the method 150, a lipid solution 152 is injected, delivered, or otherwise directed into a first inlet 114A of the plurality of inlets 114 of the fluidic mixing unit 104A. A cargo nucleic acid 154, which in this example is mRNA, is injected, delivered, or otherwise directed into the remaining inlets 114B, 114C, 114D of the plurality of inlets 114 of the fluidic mixing unit 104A. The steps 150A, 150B of injecting the lipid solution 152 and injecting the mRNA 154 are performed simultaneously. Further, in an embodiment and as described in more detail herein with respect to FIGS. 13A-13I, the steps 150A, 150B of injecting the lipid solution 152 and injecting the mRNA 154 are performed via a pipetting robot having a plurality of pipettes.

In an embodiment, before injection into the fluidic mixing unit 104A, the mRNA 154 is dissolved in an aqueous buffer solution. Similarly, before injection into the fluidic mixing unit 104A, the lipid solution 152 may include at least one lipid dissolved in a lower alcohol or an organic solvent such as but not limited to ethanol. In an embodiment, the lipid solution 152 includes at least an ionizable or cationic lipid, a cholesterol, a phospholipid or other helper lipid, and a PEG lipid. For example, the ionizable lipid may constitute between 45-55% of the lipid solution, the cholesterol may constitute between 32-42%, the phospholipid may constitute between 5-15% of the lipid solution, and the PEG lipid may constitute between 1-3% of the lipid solution. The types and relative amounts of lipids influence nanoparticle size, surface properties and encapsulation efficiency and release of the mRNA.

With reference to step 150C of the method 150, the lipid solution 152 and the mRNA 154 mix or combine within the mixing channel 116 of the fluidic mixing unit 104A for a time sufficient to form at least one lipid nanoparticle. In an embodiment, the lipid solution 152 and the mRNA 154 mix or combine within the mixing channel 116 of the fluidic mixing unit 104A to form a plurality of lipid nanoparticles of the same type or formulation. Although it is believed that the lipid solution 152 and the mRNA 154 primarily mix or combine within the mixing channel 116 adjacent to or near the junction 124, the lipid solution 152 and the mRNA 154 may mix or combine at any point along the length of the mixing channel 116. As described above, each of the four fluid inlet channels 112 of the fluidic mixing unit 104A have the same shape and size. Forming the four fluid inlet channels 112 of the same size and shape may result in a more consistent delivery of the material components into the mixing channel 116, which thereby results in more uniform mixing of the material components within the mixing channel 116. In addition, in lipid nanoparticle production, forming the four fluid inlet channels 112 of the same size and shape preserve the ratio between ethanolic and aqueous solutions. For lipid nanoparticle production, it has been determined that 3:1 is a desirable ratio of material components for mRNA to lipid solution. However, the number of fluid inlet channels 112 is exemplary and may vary depending on the material components and the particular application. The fluidic mixing unit 104A may include a lesser number or greater number of fluid inlet channels 112 in order to vary the ratio of mRNA to lipid solution or include additional components. As a non-limiting example, according to another embodiment hereof (not shown), the fluidic mixing unit 104A may include only three fluid inlet channels 112, such that the lipid solution 152 is injected, delivered, or otherwise directed into the first inlet 114A of the plurality of inlets 114 and the mRNA 154 is injected, delivered, or otherwise directed into the remaining inlets 114B, 114C of the plurality of inlets 114, if it is determined that 2:1 is a desirable ratio for mRNA to lipid solution. As another non-limiting example, according to another embodiment hereof (not shown), the fluidic mixing unit 104A may include four fluid inlet channels 112, with one of the fluid inlet channels being configured to join the mixing channel 116 downstream of the junction 124. In such an example, the lipid solution is injected, delivered, or otherwise directed into a first inlet of the plurality of inlets and the mRNA is injected, delivered, or otherwise directed into second and third inlets of the plurality of inlets. Another fluid is injected, delivered, or otherwise directed into the fourth inlet and such fluid provides in-line dilution added after particle formation.

When the lipid solution 152 and the mRNA 154 mix or combine within the mixing channel 116, lipid nanoparticles are formed through lipid reorganization and hydrophobic ion pairing of the mRNA and the ionizable lipid of the lipid solution 152. The lipid nanoparticles are composed of a combination of lipids that encapsulate the mRNA. In order to stably encapsulate the mRNA, the molecule has to interact with the ionizable lipid of the lipid solution 152 and together with the helper lipid, the PEG-lipid and cholesterol, the lipid nanoparticles are formed. The ionizable lipid of the lipid solution 152 interacts with the negative mRNA as the polarity of the solution increases. As the polarity increases over time the helper lipids, cholesterol and PEG-lipids build up the particle structure. The role of the helper lipid and the cholesterol is to build up the general architecture of the lipid nanoparticles, while keeping the surface uncharged. The function of the PEG-lipid is to reduce the aggregation of the lipid nanoparticles and control the size of the lipid nanoparticles and biodistribution.

With reference to step 150D of the method 150, after mixing of the material components within the mixing channel 116, lipid nanoparticles formed exits the single outlet 118 of the fluidic mixing unit 104A.

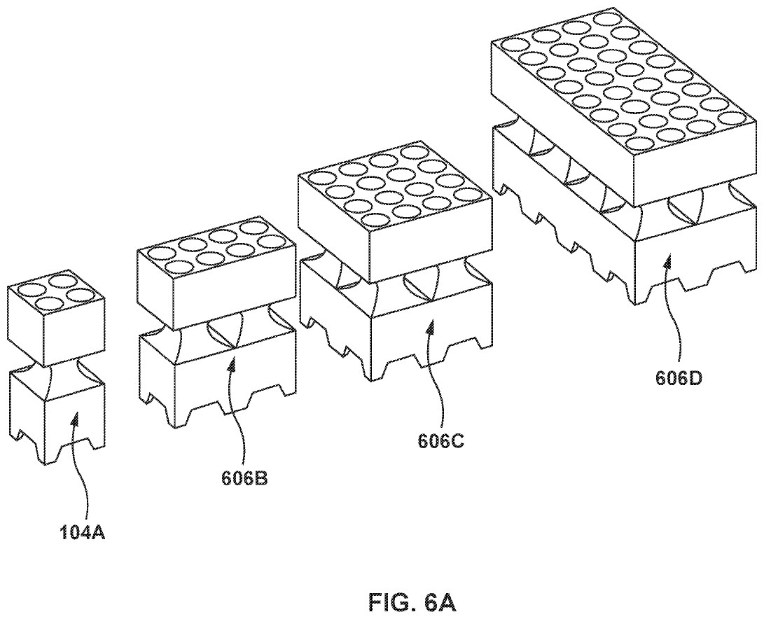

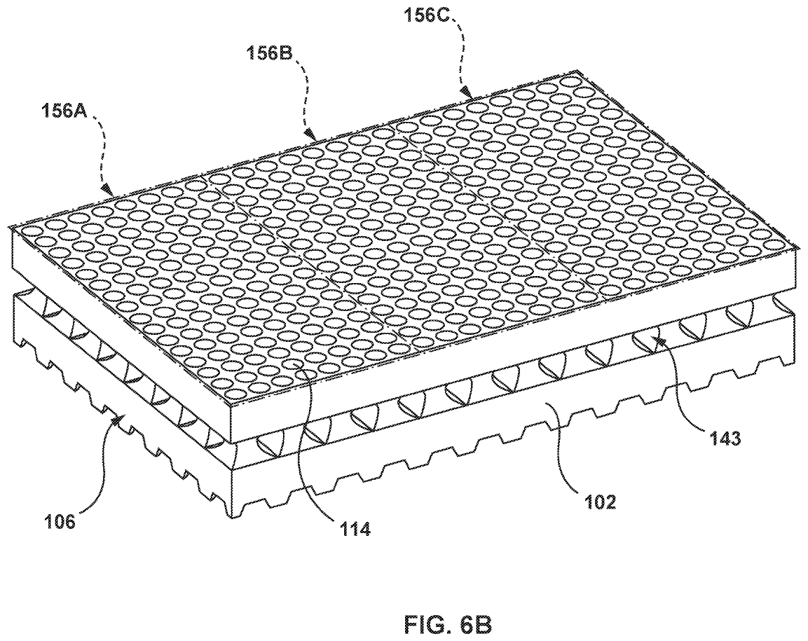

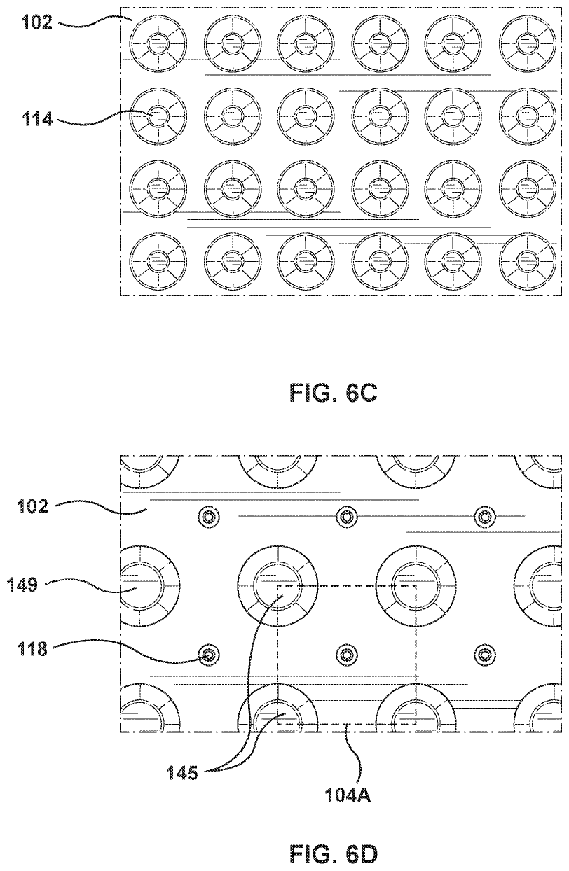

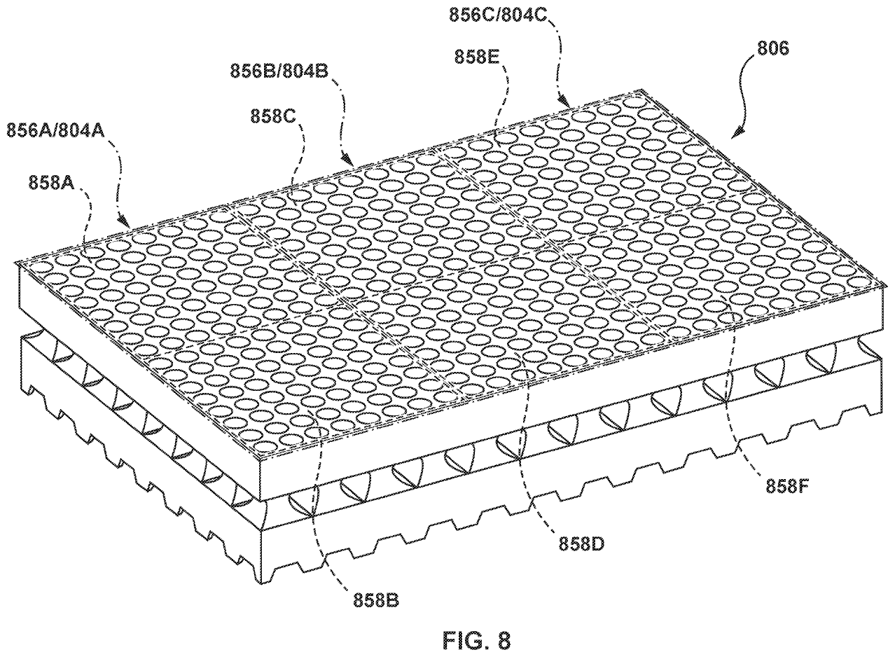

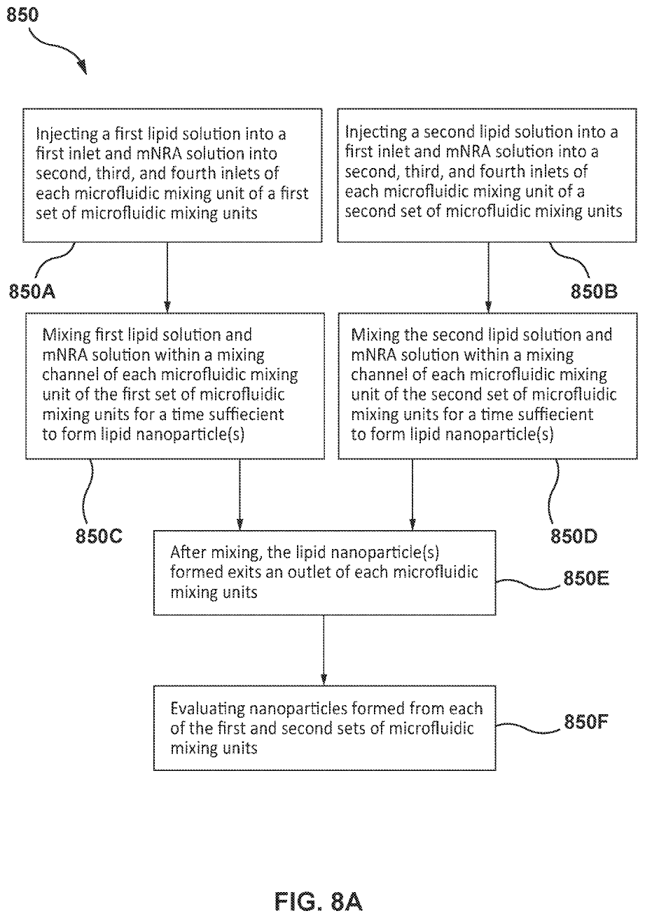

To better illustrate how multiple fluidic mixing units 104 are joined in an array to form the microplate 102, FIG. 6A illustrates a progression of the fluidic mixing unit 104A when additional fluidic mixing units 104 are added thereto. FIG. 6A illustrates an array 606B including two fluidic mixing units, an array 606C including four fluidic mixing units, and an array 606D including eight fluidic mixing units. FIG. 6B illustrates the microplate 102 including the array 106 of ninety-six fluidic mixing units 104. In an embodiment, each fluidic mixing unit of the array 106 is identical to the fluidic mixing unit 104A having the first configuration 130 of the flowpath 110. However, as will be apparent from the description below relating to FIGS. 8-12, it is not required that all fluidic mixing units of the array 106 have the same flowpath configuration. When including ninety-six fluidic mixing units, the microplate 102 includes a total of three hundred and eighty four inlets 114 and a total of ninety-six outlets 118. FIG. 6C illustrates an enlarged view of a top surface of a portion of the microplate 102 to better illustrate the pattern of the inlets 114. Similarly, FIG. 6D illustrates an enlarged view of a bottom surface of a portion of the microplate 102 to better illustrate the pattern of the outlets 118. However, the number of fluidic mixing units within the array 106 is exemplary and may vary. The array 106 may include a lesser number or greater number of fluidic mixing units. In an embodiment, the microplate 102 includes between 50 and 100 fluidic mixing units.

As described in more detail herein, in an embodiment, the number of fluidic mixing units 104 of the array 106 is compatible with or corresponds to a number of pipetting channels used by commercially available pipetting robots. More particularly, the inlets 114 of the microplate 102 are disposed in a pattern that is configured to be compatible with a pipetting robot having a plurality of pipettes. In an embodiment, the pattern of inlets 114 is a two-dimensional rectangular grid with predetermined spacing between adjacent inlets. The spacing between adjacent inlets is configured to mate with or correspond to a spacing of the plurality of pipettes of the pipetting robot. Each inlet 114 of the microplate 102 is configured to receive fluid from a respective pipette of the plurality of pipettes.

As shown on FIG. 1, the microplate 102 may be considered to include a first or top or inflow level 146, a second or bottom or outflow level 148 on an opposite side of the microplate from the first level 146, and an intermediate level 147 extending between the first level 146 and the second level 148. The first level 146 includes the inflow end portions 140 of the fluidic mixing units 104, the second level 148 includes the outflow end portions 142 of the fluidic mixing units 104, and the intermediate level 147 includes the waist portions 144 of the fluidic mixing units 104. Accordingly, the first level 146 includes the inlets 114 of the microplate 102 and the second level 148 includes the outlets 118 of the microplate 102. The microplate 102 may alternatively be considered to be a microtiter plate, a microwell plate or a multiwell plate.

As stated above, the waist portion 144 of each fluidic mixing unit 104A has a reduced perimeter relative to the first end portion 140 and the second end portion 142. With respect to the microplate 102, the reduced perimeters of the waist portions 144 of the fluidic mixing units 104 collectively form conduits 143 within the microplate 102 as shown on FIG. 6B. The conduits 143 are openings that may be configured to receive one or more cables or pipes therethrough to control temperature throughout the microplate 102, as temperature may be an extra parameter in lipid nanoparticle formation. In addition to temperature control, the conduits 143 may be used to receive a tool or fork lift to assist in disengaging the microplate 102 from a pipetting robot (described in more detail with respect to FIGS. 13A-13I). Lastly, the conduits 143 reduce the amount of resin used to print the microplate and thereby reduce the manufacturing cost thereof.

As shown on FIG. 6D, the bottom surface of the microplate 102 includes a plurality of columns 149. Each column 149 is integrally formed by the corner posts 145 of neighboring or adjacent fluidic mixing units 104. The plurality of columns 149 function to recess the bottom surface of the microplate 102 such that the microplate 102 is configured to mate with a well plate that collects the formed lipid nanoparticles. More particularly, with reference to FIG. 7, the plurality of columns 149 are configured to mate with and be received with a plurality of openings or holes 762 of a well plate 760. The mating relationship between the plurality of columns 149 and the plurality of holes 762 stabilize the well plate 760 during production of the lipid nanoparticles. The well plate 760 also includes a plurality of wells 764 that are aligned with the outlets 118 of the microplate 102 and are configured to collect or receive the formed lipid nanoparticles. Use of the well plate 760, along with a pipetting robot, is illustrated in the method depicted in FIGS. 13A-13I. As described in more detail below with respect to FIGS. 13A-131, the pipetting robot and the well plate 760 should correspond or match such that they are configured for use together. In the embodiment of FIG. 7, the well plate 760 is custom made, i.e., designed and 3D printed for use with a particular pipetting robot. In another embodiment, as depicted in FIG. 7A, a well plate 760A for receiving the formed lipid nanoparticles may be a standard and commercially available 96-well plate when the pipetting robot is a 384-channel pipetting robot. The well plate 760A includes a plurality of openings or holes 762A that receive the plurality of columns 149. The well plate 760A also includes a plurality of wells 764A, formed between the holes 762A, for receiving the formed lipid nanoparticles.

Advantageously, since the microplate 102 includes the array 106 of fluidic mixing units 104, different lipid solutions may be injected into a plurality of fluidic mixing units 104 to evaluate the effect of the variations on lipid nanoparticle production. As stated above, the types and relative amounts of lipids within the lipid solution 152 influence particle size, surface properties and encapsulation efficiency of the mRNA. For example, as shown on FIG. 6B, the array 106 of fluidic mixing units 104 may be considered to include three sets 156A, 156B, 156C of fluidic mixing units 104. A first lipid solution may be injected into the first set 156A of fluidic mixing units, a second lipid solution may be injected into the second set 156B of fluidic mixing units 104, and a third lipid solution may be injected into the third set 156C of fluidic mixing units 104. The first lipid solution is different from each of the second lipid solution and the third lipid solution, and the second lipid solution is different from the third lipid solution. As used herein, a lipid solution is considered different from another lipid solution when it includes at least one of a different lipid type or material and/or a different amount or concentration of a lipid type or material. In an embodiment, all fluidic mixing units 104 of the microplate 102 are of the same configuration (i.e., having the same flowpath configuration or design) and different lipid solutions are injected into a plurality of fluidic mixing units 104 to evaluate the effect of the variations on lipid nanoparticle production.

In addition to providing the ability to simultaneously evaluate different lipid solutions, the microplate 102 also provides the ability to simultaneously evaluate different configurations or designs of the flowpaths of the fluidic mixing units. Stated another way, the physical design or geometry of the flowpaths of the fluidic mixing units is an experimental parameter which can be varied in addition to or as an alternative to different lipid solutions to evaluate the effect of the variations on lipid nanoparticle production. With reference to FIG. 8, in another embodiment, an array 806 of fluidic mixing units may be considered to include three sets 856A, 856B, 856C of fluidic mixing units 804A, 804B, 804C, respectively. The first set 856A of fluidic mixing units 804A has a first configuration or design of the flowpath of each fluidic mixing unit thereof, the second set 856B of fluidic mixing units 804B has a second configuration or design of the flowpath of each fluidic mixing unit thereof, and the third set 856C of fluidic mixing units 804C has a third configuration or design of the flowpath of each fluidic mixing unit thereof. The first configuration is different from each of the second configuration and the third configuration, and the second configuration is different from the third configuration. As used herein, a configuration or design of a flowpath is considered different from another configuration or design of a flowpath when it includes at least one of a different geometry or shape and/or a different size or dimension.

In an embodiment, different lipid solutions may simultaneously be evaluated along with the different configurations of the flowpaths of the fluidic mixing units. More particularly, two or more different lipid solutions may be injected into subsets of the three sets 856A, 856B, 856C of fluidic mixing units 804A, 804B, 804C. For example, a first lipid solution may be injected into a first subset 858A of the fluidic mixing units 804A of the first set 856A and a second lipid solution which is different from the first lipid solution may be injected into a second subset 858B of the fluidic mixing units 804A of the first set 856A. The first lipid solution may also be injected into a first subset 858C of the fluidic mixing units 804B of the second set 856B and the second lipid solution may be injected into a second subset 858D of the fluidic mixing units 804B of the second set 856B. The first lipid solution may also be injected into a first subset 858E of the fluidic mixing units 804C of the third set 856C and the second lipid solution may be injected into a second subset 858F of the fluidic mixing units 804C of the third set 856C. This exemplary set-up permits simultaneous evaluation of different combinations of lipid solutions and flowpath geometries to compare the effect of the variations on lipid nanoparticle production. It will be apparent to one of ordinary skill in the art that the exemplary set-up described with respect to FIG. 8 is one of countless combinations of flowpath geometries and lipid solutions which may be simultaneously tested on an array of a single device.

With reference to the flow chart of FIG. 8A, a method 850 of evaluating lipid nanoparticle production with a device according to embodiments hereof is described in more detail. In the method 850, the device includes a microplate including an array of a plurality of fluidic mixing units and the plurality of fluidic mixing units includes a first set of fluidic mixing units and a second set of fluidic mixing units.

With reference to step 850A of the method 850, a first lipid solution is injected, delivered, or otherwise directed into a first inlet and mRNA into second, third, and fourth inlets of each fluidic mixing unit of the first set of fluidic mixing units. With reference to step 850B of the method 850, a second lipid solution is injected, delivered, or otherwise directed into a first inlet and mRNA into second, third, and fourth inlets of each fluidic mixing unit of the second set of fluidic mixing units. The injecting steps 850A, 850B are performed substantially simultaneously. Further, in an embodiment and as described in more detail herein with respect to FIGS. 13A-13I, the injecting steps 850A, 850B are performed via a pipetting robot having a plurality of pipettes. In an embodiment, the first lipid solution is different from the second lipid solution. In another embodiment, the first set of fluidic mixing units has a first configuration or design of the flowpath, the second set of fluidic mixing units has a second configuration or design of the flowpath, and the first configuration is different from the second configuration.

As described above with respect to the method 150, each of the first and second lipid solutions includes at least one lipid dissolved in a lower alcohol or an organic solvent such as but not limited to ethanol. In an embodiment, each of the first and second lipid solutions includes an ionizable or cationic lipid, a cholesterol, a phospholipid or helper lipid, and a PEG lipid. The mRNA is dissolved in an aqueous buffer solution.

With reference to step 850C of the method 850, the first lipid solution and mRNA mix or combine within the mixing channel of each fluidic mixing unit of the first set of fluidic mixing units for a time sufficient to form lipid nanoparticles. With reference to step 850D of the method 850, the second lipid solution and mRNA mix or combine within the mixing channel of each fluidic mixing unit of the second set of fluidic mixing units for a time sufficient to form lipid nanoparticles. The mixing steps 850C, 850D are performed substantially simultaneously. As described above with respect to the method 150, when the respective lipid solution and the mRNA mix or combine within the mixing channel, lipid nanoparticles are formed through lipid reorganization and hydrophobic ion pairing of the mRNA and the ionizable lipid of the lipid solution. With reference to step 850E of the method 850, after mixing of the material components within the mixing channel, lipid nanoparticles formed exits the outlet of each fluidic mixing unit.

With reference to step 850F, the lipid nanoparticles formed from each set of fluidic mixing units are evaluated. More particularly, at least one parameter of the lipid nanoparticles is evaluated. The parameter may be, for example, a size of the nanoparticles, a surface charge of the nanoparticles and an encapsulation efficiency of the nanoparticles. In addition, the method may further include comparing the nanoparticles formed from the first set of fluidic mixing units with the nanoparticles formed from the second set of fluidic mixing units.

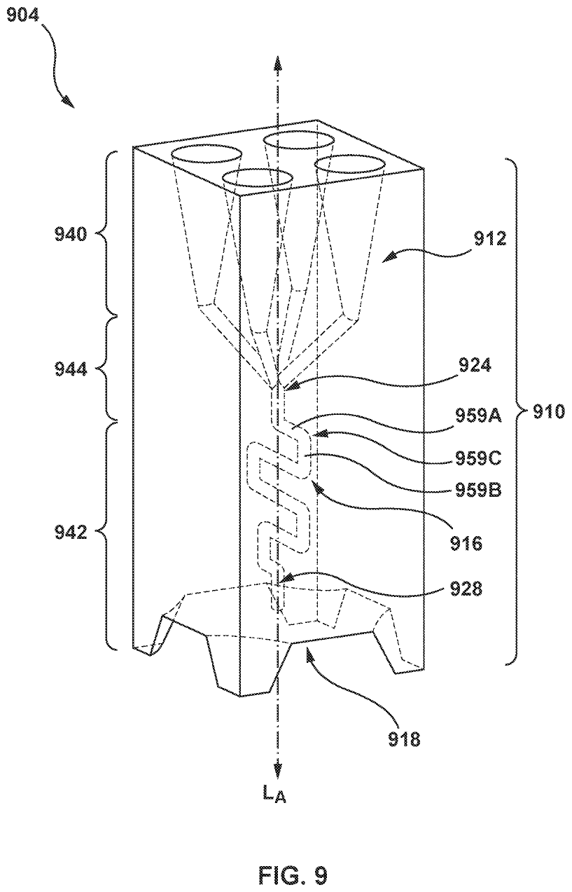

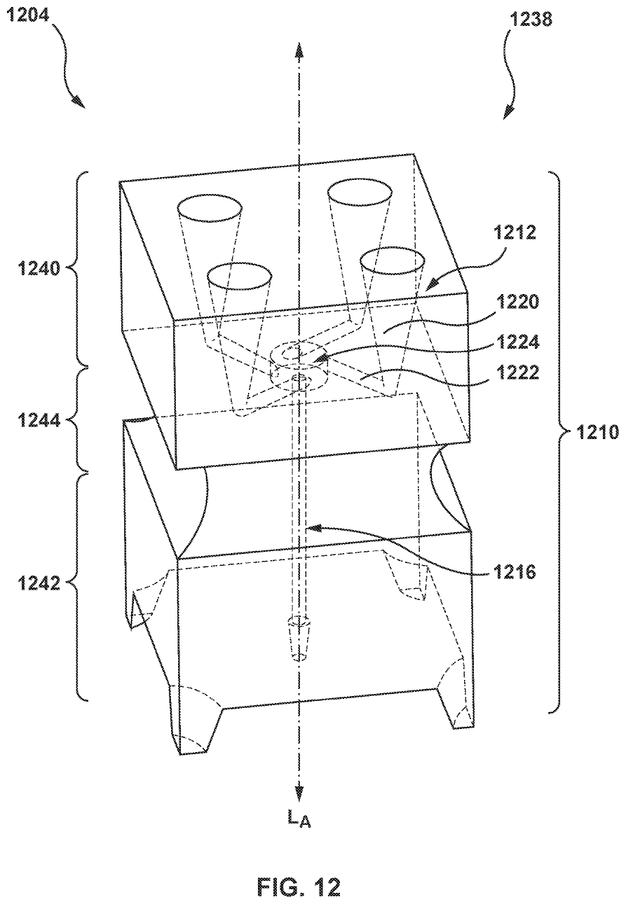

While not intending to be limited to the examples described herein, FIGS. 9-12 illustrate exemplary configurations of flowpaths for fluidic mixing units 804A, 804B, 804C described above with respect to FIG. 8. More particularly, FIGS. 9-11 depict alternative configurations of the mixing channel as compared to the mixing channel 116 of the fluidic mixing unit 104A and FIG. 12 depicts an alternative configuration of the cylindrical portions of the fluid inlet channels and the junction as compared to the cylindrical portions 122 of the fluid inlet channels 112 and the junction 124 of the fluidic mixing unit 104A.

More particularly, FIG. 9 is a semi-transparent perspective view of a single fluidic mixing unit showing the flowpath thereof, which is labeled and referred to herein as fluidic mixing unit 904. The fluidic mixing unit 904 is configured to produce one type or formulation of lipid nanoparticles. The fluidic mixing unit 904 includes a first or inflow end portion 940 and a second or outflow end portion 942 opposing the first end portion 940 and a waist portion 944 extending between the first end portion 940 and the second end portion 942. In this embodiment, the waist portion 944 has the same perimeter as the first end portion 940 and the second end portion 942. Although the waist portion 144 of the fluidic mixing unit 104 having a reduced perimeter has certain advantages as described above, the reduced perimeter is not required as the outer geometry of the fluidic mixing unit 904 does not affect the operation of the internal geometry or flowpath.

The fluidic mixing unit 904 has a second configuration or geometry 932 of a flowpath 910 thereof. The flowpath 910 extends between the top and bottom surfaces of the microplate 102. The second configuration 932 of the flowpath 910 is different from the first configuration 130 of the flowpath 110 of the fluidic mixing unit 104A. The second configuration 932 includes four fluid inlet channels 912. In this embodiment, the fluid inlet channels 912 of the second configuration 932 are the same as the fluid inlet channels 112 of the first configuration 130. At a junction 924, the four fluid inlet channels 912 converge or transition into a mixing channel 916. In the second configuration 932 of the flowpath 910, the mixing channel 916 has a series of alternating transverse portions 959A and longitudinal portions 959B, with corners 959C therebetween and an exit or tip portion 928. The alternating portions 959A, 959B extend between the junction 924 and the tip portion 928, and the tip portion 928 extends between the alternating portions 959A, 959B and an outlet 918 of the mixing channel 916. The transverse portions 959A extend perpendicular or substantially perpendicular to a longitudinal axis L_{A} of the fluidic mixing unit 904 and the longitudinal portions 959B extend parallel or substantially parallel to the longitudinal axis L_{A} of the fluidic mixing unit 904. Each transverse portion 959A crosses the longitudinal axis L_{A} as it extends between the two longitudinal portions 959B adjacent thereto. In an embodiment, the alternating portions 959A, 959B extend in a single plane through the fluidic mixing unit 904. The alternating portions 959A, 959B have a constant diameter along a length thereof, which may range between 300 μm and 800 μm. The tip portion 928 extends parallel or substantially parallel to the longitudinal axis L_{A} of the fluidic mixing unit 904. In an embodiment, the tip portion 928 has a diameter which is greater than the diameter of the alternating portions 959A, 959B. The diameter of the tip portion 928 may range between 300 μm and 1000 μm, and may be constant along a length thereof or may be varied.

FIG. 10 is a semi-transparent perspective view of a single fluidic mixing unit showing the flowpath thereof, which is labeled and referred to herein as fluidic mixing unit 1004. The fluidic mixing unit 1004 is configured to produce one or more lipid nanoparticles. The fluidic mixing unit 1004 includes a first or inflow end portion 1040, a second or outflow end portion 1042 opposing the first end portion 1040 and a waist portion 1044 extending between the first end portion 1040 and the second end portion 1042. The fluidic mixing unit 1004 has a third configuration or geometry 1034 of a flowpath 1010 thereof, that is different that each of the second configuration 932 of the flowpath 910 and the first configuration 130 of the flowpath 110. The flowpath 1010 extends between the top and bottom surfaces of the microplate 102. The third configuration 1034 includes four fluid inlet channels 1012. In this embodiment, the fluid inlet channels 1012 of the third configuration 1034 are the same as the fluid inlet channels 112 of the first and second configurations 130, 932. At a junction 1024, the four fluid inlet channels 1012 converge or transition into a mixing channel 1016. In the third configuration 1034 of the flowpath 1010, the mixing channel 1016 has a wavy portion 1059 and an exit or tip portion 1028. The wavy portion 1059 extends between the junction 1024 and the tip portion 1028, and the tip portion 1028 extends between the wavy portion 1059 and an outlet 1018 of the mixing channel 1016. The wavy portion 1059 includes a plurality of alternating or oscillating curves, similar to a sine wave, along a length thereof. In an embodiment, the wavy portion 1059 extends in a single plane through the fluidic mixing unit 1004. The wavy portion 1059 has a constant diameter along a length thereof, which may range between 300 μm and 800 μm. The tip portion 1028 extends parallel or substantially parallel to a longitudinal axis L_{A} of the fluidic mixing unit 1004. In an embodiment, the tip portion 1028 has a diameter which is greater than the diameter of the wavy portion 1059. The diameter of the tip portion 1028 may range between 300 μm and 1000 μm, and may be constant along a length thereof or may be varied.

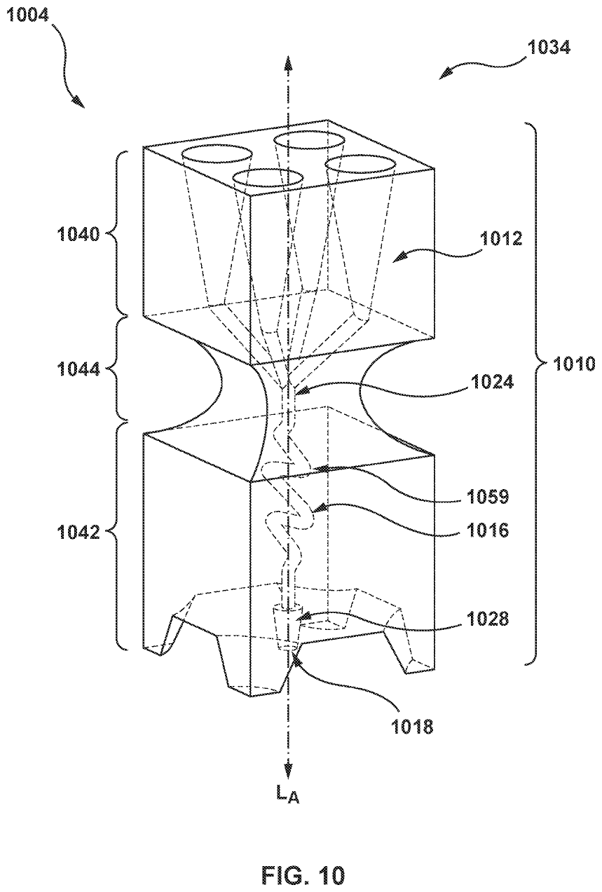

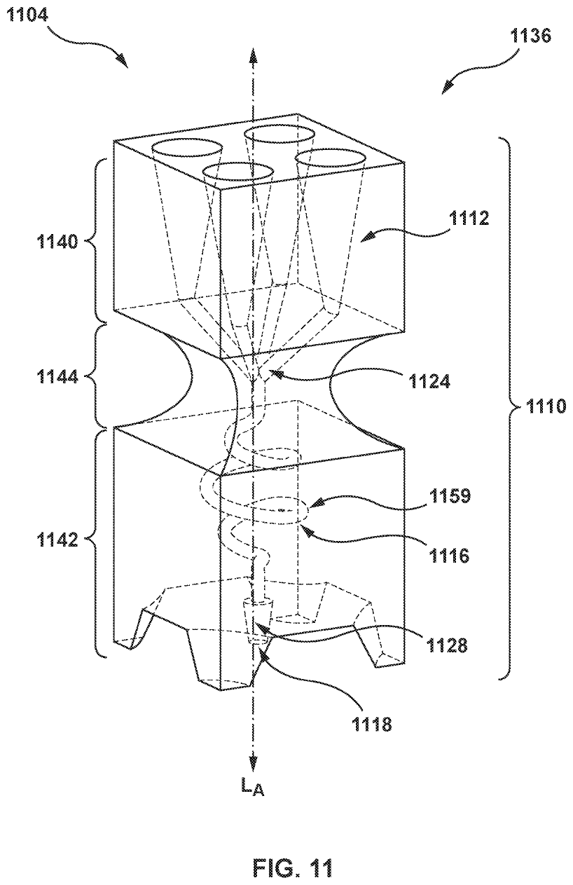

FIG. 11 is a semi-transparent perspective view of a single fluidic mixing unit showing the flowpath thereof, which is labeled and referred to herein as fluidic mixing unit 1104. The fluidic mixing unit 1104 is configured to produce one or more lipid nanoparticles. The fluidic mixing unit 1104 includes a first or inflow end portion 1140, a second or outflow end portion 1142 opposing the first end portion 1140 and a waist portion 1144 extending between the first end portion 1140 and the second end portion 1142. The fluidic mixing unit 1104 has a fourth configuration or geometry 1136 of a flowpath 1110 thereof that is different than each of the first, second and third configurations 130, 932, 1034 of the flowpaths described above. The flowpath 1110 extends between the top and bottom surfaces of the microplate 102. The fourth configuration 1136 includes four fluid inlet channels 1112. In this embodiment, the fluid inlet channels 1112 of the fourth configuration 1136 are the same as the fluid inlet channels 112 of each of the previously described configurations. At a junction 1124, the four fluid inlet channels 1112 converge or transition into a mixing channel 1116. In the fourth configuration 1136 of the flowpath 1110, the mixing channel 1116 has a spiral or coiled portion 1159 and an exit or tip portion 1128. The spiral portion 1159 extends between the junction 1124 and the tip portion 1128, and the tip portion 1128 extends between the spiral portion 1159 and an outlet 1118 of the mixing channel 1116. The spiral portion 1159 includes a plurality of spaced-apart windings along a length thereof. The spiral portion 1159 has a constant diameter along a length thereof, which may range between 300 μm and 800 μm. The tip portion 1128 extends parallel or substantially parallel to a longitudinal axis L_{A} of the fluidic mixing unit 1104. In an embodiment, the tip portion 1128 has a diameter which is greater than the diameter of the spiral portion 1159. The diameter of the tip portion 1128 may range between 300 μm and 1000 μm, and may be constant along a length thereof or may be varied.

FIG. 12 is a semi-transparent perspective view of a single fluidic mixing unit showing the flowpath thereof, which is labeled and referred to herein as fluidic mixing unit 1204. The fluidic mixing unit 1204 is configured to produce one or more lipid nanoparticles. The fluidic mixing unit 1204 includes a first or inflow end portion 1240, a second or outflow end portion 1242 opposing the first end portion 1240 and a waist portion 1244 extending between the first end portion 1240 and the second end portion 1242. The fluidic mixing unit 1204 has a fifth configuration or geometry 1238 of a flowpath 1210 thereof that is different from each of the previously described configurations. The flowpath 1210 extends between the top and bottom surfaces of the microplate 102. The fifth configuration 1238 includes four fluid inlet channels 1212. In this embodiment, a mixing channel 1216 of the fifth configuration 1238 is the same as the mixing channel 116 of the first configuration 130 and generally cone-shaped portions 1220 of the fluid inlet channels 1212 of the fifth configuration 1238 are the same as the generally cone-shaped portions 120 of the fluid inlet channels 112 of the first configuration 130. However, in this embodiment, cylindrical portions 1222 of the fluid inlet channels 1212 extend perpendicular or substantially perpendicular relative to a longitudinal axis L_{A} of the fluidic mixing unit 1204. The cylindrical portions 1222 of each fluid inlet channel 1212 has a constant diameter along a length thereof, which may range between 300 μm and 800 μm. At a junction 1224, the four fluid inlet channels 1212 converge or transition into the mixing channel 1216. In this embodiment, the junction 1224 has an enlarged diameter relative to the diameter of the junction 124 of the first configuration 130. The diameter of junction 1224 may be between 3 and 6 times larger than the diameter of the cylindrical portions 1222 of each fluid inlet channel 1212.

As stated above, the device 100 is configured to be compatible with an automation platform and a commercially available pipetting or dispensing robot. FIGS. 13A-13I depict a method of using the device 100 with a pipetting robot 1370 and an automated platform in which fluid material components are pipetted from a source plate to the device 100 where they are rapidly mixed within the plurality of fluidic mixing units 104 and formed nanoparticles are collected directly into the well plate 760A for further use. In the depicted embodiment of FIGS. 13A-13I, the pipetting robot 1370 is a 384-channel Bravo pipetting robot commercially available from Agilent. However, the device 100 may be designed to be compatible with others pipetting robot as desired. The pipetting robot and the well plate should correspond or match such that they are configured for use together. When the pipetting robot 1370 is a 384-channel pipetting robot, the well plate 760A described above with respect to FIG. 7A may be utilized for receiving the formed lipid nanoparticles. As described above, the well plate 760A is a standard and commercially available 96-well plate, such as a 96-well plate commercially available from Greiner Bio-One of North Carolina, USA. Alternatively, if another pipetting robot is utilized which requires a non-standard recipient well plate, a custom made well plate, such as the well plate 760 described above with respect to FIG. 7, may be utilized for receiving the formed lipid nanoparticles.

FIGS. 13A-13C depict the pipetting robot 1370 being connected to a plurality of pipette tips 1372. More particularly, FIG. 13A illustrates the pipetting robot 1370 disposed above the plurality of pipette tips 1372. In FIG. 13B, the pipetting robot 1370 is lowered into contact with the plurality of pipette tips 1372 so that the plurality of pipette tips 1372 may attach to the pipetting robot 1370. In FIG. 13C, the pipetting robot 1370 is raised after the plurality of pipette tips 1372 are attached thereto.

FIGS. 13D-13F depict the plurality of pipette tips 1372 being filled with fluid substances. More particularly, the pipetting robot 1370 is moved to a position in which the pipetting robot 1370 disposed above a source plate 1374 as shown in FIG. 13D. As will be understood by those of ordinary skill in the art, the source plate 1374 includes a plurality of containers which are filled with fluid material components to be delivered into the device 100, i.e., lipid solution(s) and mRNA. The spacing between adjacent containers is configured to mate with or correspond to a spacing of the plurality of pipette tips of the pipetting robot, so that each pipette tip 1372 may receive fluid from a container/well of the source plate 1374. In FIG. 13E, the pipetting robot 1370 is lowered such that the plurality of pipette tips 1372 are disposed within the plurality of containers of the source plate 1374 to enable filling of the pipette tips (fluid aspiration). In FIG. 13F, the pipetting robot 1370 is raised after the plurality of pipette tips 1372 are filled with the fluid material components to be delivered into the device 100, i.e., lipid solution(s) and mRNA. The pipette tips 1372 are depicted as cloudy or opaque in FIG. 13F and FIG. 13G as they are filled with the fluid material components to be delivered into the device 100, i.e., lipid solution(s) and mRNA.

FIGS. 13G-13I depict the fluid within the plurality of pipette tips 1372 being delivered into the device 100. More particularly, the pipetting robot 1370 is moved to a position in which the pipetting robot 1370 disposed above the device 100 as shown in FIG. 13G. The device 100 is disposed on top of the well plate 760A, which includes the plurality of wells 764A that are aligned with the outlets 118 of the microplate 102 and are configured to collect or receive the formed lipid nanoparticles as described above with respect to FIG. 7. In FIG. 13H, the pipetting robot 1370 is lowered such that the tips of the plurality of pipette tips 1372 are disposed within the inlets 114 of the microplate 102 of the device 100. As previously stated, the inlets 114 of the microplate 102 are disposed in a pattern that is configured to be compatible with the plurality of pipette tips 1372 of the pipetting robot 1370 such that each inlet 114 of the microplate 102 receives fluid from a pipette of the plurality of pipette tips 1372. As described above, the inlets 114 of the microplate 102 are configured to receive and mate with tips of the plurality of pipette tips 1372. Engagement between the plurality of pipette tips 1372 and the inlets 114 may be optimized by the design of the inlet geometry such that the tips of the pipette tips 1372 extend into the fluid inlet channels 112 a sufficient amount to create an adequate seal and prevent leakage, but do not extend so far into the fluid inlet channels 112 that the pipette tips pinch and possibly block the fluid inlet channels 112. Proper engagement between the plurality of pipette tips 1372 and the inlets 114 ensures that all reagents will fluidly pass from the pipette tips 1372 and through the fluid inlet channels 112 of the device 100.

The dispensing flow rate of the pipetting robot 1370 is also an experimental variable or parameter that may affect quality of the lipid nanoparticles. Further, it is plausible that the actual flow rate achieved may not reach the set or programmed value because the pipetting robot 1370 does not achieve an instantaneous velocity and each pipette tip has a relatively small capacity. Depending on injection volume, in an embodiment, the actual flow rate within the device 100 ranges between 0.1 and 500 μL/sec.

In FIG. 13I, the pipetting robot 1370 is raised after the fluid material components, i.e., lipid solution(s) and mRNA, are delivered into the device 100. As can be seen in FIG. 13I, when the pipetting robot 1370 is raised after fluid delivery, the device 100 is still coupled to the plurality of pipette tips 1372 via engagement between the pipette tips and the inlets 114 of the microplate 102. The well plate 760A and lipid nanoparticles received therein are therefore exposed for inspection and further use. The output from multiple devices 100 may be pooled in order to scale-up the formulation volume in contexts where larger formulation volumes are necessary (e.g., in-vivo animal experiments).

For any embodiment described herein, the microplate may be constructed of a resin material and may be formed via 3D printing such that the plurality of fluidic mixing units are integrally formed with the microplate. In an embodiment, the microplate may be constructed of a clear or transparent material to permit visibility of the fluid material components therein. Ceramic-like materials that can be sterilized or materials compatible with clinical applications may also be used. The 3D printer preferably has characteristics including low cost, good safety, high volumetric resolution, reasonable speed and the ability to work with materials that are compatible with both nanomedicine and biological components. Any 3D printer with these characteristics may be used to produce the microplates described herein. For example, Phrozen Sonic Mini 4K is a 3D printer that is suitable for 3D printing the microplates described herein.

With reference to the flow chart of FIG. 14, a method 1480 of manufacturing a device according to embodiments hereof is described in more detail. In the method 1480, the device includes a microplate including an array of a plurality of fluidic mixing units and the plurality of fluidic mixing units includes a first set of fluidic mixing units and a second set of fluidic mixing units. The first set of fluidic mixing units has a first configuration or design of the flowpath, the second set of fluidic mixing units has a second configuration or design of the flowpath, and the first configuration is different from the second configuration.

The method 1480 is computer-implemented by at least one processor executing software instructions. A computing device useful for practicing the method 1480 may include, without limitation, a processor, a memory unit, and a storage device including an operating system and software. The processor is any logic circuity that responds to and processes instructions fetched from the memory unit. In many embodiments, the processor is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER4 processor, those manufactured by International Business Machines of White Plains, N.Y.; or those manufactured by Advanced Micro Devices of Sunnyvale, Calif. The computing device may be based on any of these processors, or any other processor capable of operating as described herein. The processor may utilize instruction level parallelism, thread level parallelism, different levels of cache, and multi-core processors. A multi-core processor may include two or more processing units on a single computing component. Examples of multi-core processors include the AMD PHENOM IIX2, INTER CORE i5 and INTEL CORE i4.

The memory unit may include on or more memory chips capable of storing data and allowing any storage location to be directly accessed by the processor. The memory unit may be volatile and faster than storage memory. The memory unit may be Dynamic Random-Access Memory (DRAM) or any variants, including static Random-Access Memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the memory unit or the storage may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The memory unit may be based on any of the above described memory chips, or any other available memory chips capable of operating as described herein.

With reference to step 1480A of the method 1480, a first fluidic mixing unit object having a first configuration or design of the flowpath is created via a software application configured for creating solid 3D computer-aided objects. In an embodiment, OpenSCAD (open source) software may be utilized to 3D draw the first fluidic mixing unit object. As used herein, “fluidic mixing unit object” refers to a computer-generated 3D object. If a fluidic mixing unit object is 3D printed by a 3D printer, the resulting printed piece corresponds to a fluidic mixing unit of the microplate.

With reference to step 1480B of the method 1480, a second fluidic mixing unit object having a second configuration or design of the flowpath is created via the software application configured for creating solid 3D computer-aided objects. The first configuration is different from the second configuration. In an embodiment, OpenSCAD (open source) software may be utilized to 3D draw the second fluidic mixing unit object.

With reference to step 1480C of the method 1480, a first set of the plurality of the first fluidic mixing unit objects and a second set of a plurality of the second fluidic mixing unit objects are assembled into an array object. In an embodiment, 3D builder software from MICROSOFT may be utilized to assemble the first set and the second set into the array object. As used herein, “array object” refers to a computer-generated 3D object. If an array object is 3D printed by a 3D printer of appropriate xy resolution capacity, the resulting printed piece corresponds to a microplate having a plurality of fluidic mixing units arranged in an array.

With reference to step 1480D of the method 1480, the device or microplate is 3D printed using the array object. In an embodiment, 3D printing the device includes generating a printing file from the array object and 3D printing the device with the printing file, the printing file having a file format suitable for 3D printing. For example, the file format of the printing file may be an STL file.

In an embodiment, the microplates described herein may be reusable. It may be desired to perform washes in between uses thereof.

It is believed that the microplates described herein, as described, are particularly useful for fluidic mixing to encapsulate different types of nucleic acids such as sgRNA, mRNA, siRNA and plasmid DNA in drug delivery systems. Microplates described herein have a significant impact in speed and cost of screening studies, such that comprehensive rich data regarding lipid nanoparticle attributes and function may be delivered very quickly to many projects and platforms for a significantly reduced cost as compared to previously known approaches. The high throughput allows testing of a wider ranges of parameters, improving confidence in the data, the data quality and mechanistic understanding.

In some embodiments, disclosed are lipid nanoparticles (LNPs) prepared by the device and/or method described herein, wherein the LNPs comprise multiple lipid components selected from ionizable lipids and helper lipids, provided that the net charge of the nanoparticle is about zero. Helper lipids are lipid molecules that increase structural stability and fluidity of lipid nanoparticles. Examples of helper lipids include neutral lipids, sterols and polymer-conjugated lipids.

Ionizable lipids are lipid molecules which remain neutral at physiological pH, but are protonated at low pH, making them positively charged. The non-limiting examples of ionizable lipids include, for instance, lipids containing a positive charge at the acidic scale of physiological pH range, for example 1,2-dilinoleyloxy-3-dimethylaminopropane (DLin-DMA), dilinoleylmethyl-4-dimethylaminobutyrate (DLin-MC3-DMA, (see e.g., U.S. Pat. No. 8,158,601), 2-dilinoleyl-4-dimethylaminoethyl-[1,3]-dioxolane (DLin-KC2-DMA), Merck-32 (see e.g., WO 2012/018754), Acuitas-5 (see e.g., WO 2015/199952), KL-10 (see e.g., U.S. Patent Application Publication 2012/0295832), C12-200 (see e.g., Love, KT et al., PNAS, 107: 1864 (2009)), and the like. The ionizable lipids may be present in an amount ranging from about 5% to about 90%, such as from about 10% to about 80%, for instance from about 25% to about 75%, for example, from about 40% to about 60%, from about 40% to about 50%, such as about 45% or about 50%, molar percent, relative to the total lipid present in the lipid nanoparticles.

The term “neutral lipid” includes lipids that have a zero-net charge at physiological pH, for example, lipids that exist in an uncharged form or neutral zwitterionic form at physiological pH, such as distearoyl phosphatidylcholine (DSPC), dioleoyl phosphatidylethanolamine (DOPE), dipalmitoyl phosphatidylcholine (DPPC), dimyristoyl phosphatidylcholine (DMPC), and the like, and combinations thereof. The neutral lipids may be present in an amount ranging from about 1% to about 50%, such as from about 5% to about 20%, for example, 7.5% to about 12.5%, for instance, about 10%, molar percent, relative to the total lipid present in the lipid nanoparticles. In some embodiments, the neutral lipid is DSPC. In some embodiments, the neutral lipid is DOPE. In some embodiments, the neutral lipid is DPPC. In some embodiments, the neutral lipid is DMPC.

The term “sterol” includes cholesterol, and the like. The sterols may be present in an amount ranging from about 10% to about 90%, such as from about 20% to about 50%, for instance, from about 35%-45%, such as about 38.5%, molar percent, relative to the total lipid present in the lipid nanoparticles. In some embodiments, the sterol is cholesterol.

The term “polymer-conjugated lipid” includes lipids that comprise a lipid portion and a polymer portion, such as pegylated lipids (also known as PEG lipids) comprising both a lipid portion and a polyethylene glycol portion. Non-limiting examples include dimyristoyl phosphatidyl ethanolamine-poly(ethylene glycol) 2000 (DMPE-PEG2000), DPPE-PEG2000, DMG-PEG2000, DPG-PEG2000, PEG2000-c-DOMG, PEG2000-c-DOPG, and the like. The molecular weight of the poly(ethylene glycol) that may be used may range from about 500 and about 10,000 Da, or from about 1,000 to about 5,000 Da. In some embodiments, the polymer-conjugated lipid is DMPE-PEG2000. In some embodiments, the polymer-conjugated lipid is DPPE-PEG2000. In some embodiments, the polymer-conjugated lipid is DMG-PEG2000. In some embodiments, the polymer-conjugated lipid is DPG-PEG2000. In some embodiments, the polymer-conjugated lipid is PEG2000-c-DOMG. In some embodiments, the polymer-conjugated lipid is PEG2000-c-DOPG. The polymer-conjugated lipids may be present in an amount ranging from about 0% to about 20%, for example about 0.5% to about 5%, such as about 1% to about 2%, for instance, about 1.5%, molar percent, relative to the total lipid present in the lipid nanoparticles.

In some embodiments of the present disclosure, lipid nanoparticles prepared by the present device and method comprise an ionizable lipid, a sterol, a neutral lipid, and a polymer-conjugated lipid at a molar ratio of 50:40-x:10:x, with respect to the total lipids present. For example, the lipid nanoparticles may be prepared combining an ionizable lipid, a sterol, a neutral lipid, and a polymer-conjugated lipid at a molar ratio of 50:37:10:3 (mol/mol), or, for instance, a molar ratio of 50:38.5:10:1.5 (mol/mol), or, for example, 50:39.5:10:0.5 (mol/mol), or 50:39.75:10:0.25 (mol/mol).

The selection of neutral lipids, sterols, and/or polymer-conjugated lipids that comprise the lipid nanoparticles, as well as the relative molar ratio of such lipids to each other, may be determined by the characteristics of the selected lipid(s), the nature of the intended target cells, and the characteristics of the nucleic acid segment to be delivered. For instance, in certain embodiments, the molar percent of the ionizable lipid in the lipid nanoparticle may be greater than about 10%, greater than about 20%, greater than about 30%, greater than about 40%, greater than about 50%, greater than about 60%, or greater than about 70%, relative to the total lipids present. The molar percent of neutral lipid in the lipid nanoparticle may be greater than about 5%, greater than about 10%, greater than about 20%, greater than about 30%, or greater than about 40%, relative to the total lipids present. The molar percent of sterol in the lipid nanoparticle may be greater than about 10%, greater than about 20%, greater than about 30%, or greater than about 40%, relative to the total lipids present. The molar percent of polymer-conjugated lipid in the lipid nanoparticle may be greater than about 0.25%, such as greater than about 1%, greater than about 1.5%, greater than about 2%, greater than about 5%, or greater than about 10%, relative to the total lipids present.

According to the present disclosure, the lipid nanoparticles may comprise each of the ionizable lipids, neutral lipids, sterols, and/or polymer-conjugated lipids in any useful orientation desired. For example, the core of the nanoparticle may comprise the ionizable lipids (which may be one species of lipid compounds or a combination of different species of lipid compounds), a sterol and one or more layers comprising neutral lipids and/or polymer-conjugated lipids may subsequently surround the core. For instance, according to one embodiment, the core of the lipid nanoparticle may comprise a core comprising an ionizable lipid, and a sterol (e.g., cholesterol) in any particular ratio, surrounded by a neutral lipid monolayer (e.g., DSPC) of any particular thickness, further surrounded by an outer polymer-conjugated lipid monolayer of any particular thickness. In such examples, the nucleic acid segment may be incorporated into any one of the core or subsequent layers depending upon the nature of the intended target cells, and the characteristics of the nucleic acid segment to be delivered. The core and outer layers may further comprise other components typically incorporated into lipid nanoparticles known in the art. Furthermore, it is understood by one skilled in the art that liposomes are delivery vehicles that possess a vesicular structure distinct from the lipid nanoparticles as disclosed herein. The liposome vesicles are composed of a lipid bilayer that forms in the shape of a hollow sphere encompassing an aqueous phase. For example, liposomes contain the lamellar phase while the lipid nanoparticles have non-lamellar structures.

In addition, the molar percent of the components of the lipid nanoparticle (e.g., the ionizable lipids, neutral lipids, sterols, and/or polymer-conjugated lipids) that comprise the lipid nanoparticles may be selected in order to provide a particular physical parameter of the overall lipid nanoparticle, such as the surface area of one or more of the lipids. For example, the molar percent of the ionizable lipids, neutral lipids, sterols, and/or polymer-conjugated lipids that comprise the lipid nanoparticles may be selected to yield a surface area per neutral lipid, for example, DSPC. By way of non-limiting example, the molar percent of the ionizable lipids, neutral lipids, sterols, and/or polymer-conjugated lipids may be determined to yield a surface area per DSPC of about 1.0 nm2 to about 2.0 nm2, for example about 1.2 nm2 .

According to the present disclosure, the lipid nanoparticles may further comprise a nucleic acid segment, which may be associated on the surface of the lipid nanoparticles and/or encapsulated within the same lipid nanoparticles.

The term “nucleic acid segment” is understood to mean any one or more nucleic acid segments selected from antisense oligonucleotides, DNA, mRNAs, siRNAs, Cas9guided-RNA complex, or combinations thereof. The nucleic acid segments herein may be wildtype or modified. In at least one embodiment, the lipid nanoparticles may comprise a plurality of different nucleic acid segments. In yet another embodiment, the nucleic acid segment, wildtype or modified, encodes a polypeptide of interest. A modified nucleic acid segment includes nucleic acid segments with chemical modifications to any part of the structure such that the nucleic acid segment is not naturally occurring. In some embodiments, the nucleic acid segment is an RNA. In some embodiments, the nucleic acid segment is an mRNA. In some embodiments, the nucleic acid segment is a modified mRNA.

The term “therapeutically effective amount” as used herein refers to an amount of nucleic acid segment sufficient to modulate protein expression in a target tissue and/or cell type. In some embodiments, a therapeutically effective amount of the nucleic acid segment is an amount sufficient to treat a disease or disorder associated with the protein expressed by the nucleic acid segment.

In at least one embodiment, the weight ratio of total lipid phase to nucleic acid segment ranges from about 40:1 to about 1:1, such as about 10:1. This corresponds to an approximate molar ratio of the ionizable lipids to nucleic acid monomer of about 3:1. In yet another example, the weight ratio of total lipid phase to nucleic acid segment ranges from about 30:1 to about 1:1, such as about 20:1, which corresponds to an approximate molar ratio of the ionizable lipids, to nucleic acid monomer of about 6:1. However, the relative molar ratio of lipid phase and/or lipid phase components to the nucleic acid monomer may be determined by the nature of the intended target cells and characteristics of nucleic acid segment and thus, are not limited in scope to the above-identified embodiments. In some embodiments, the molar ratio of the ionizable lipids to nucleic acid monomer is from about 2.75:1 to 6:1. In some embodiments, the molar ratio of the ionizable lipids to nucleic acid monomer is about 2.75:1. In some embodiments, the approximate molar ratio of the ionizable lipids to nucleic acid monomer of about 3:1. In some embodiments, the molar ratio of the ionizable lipids to nucleic acid monomer is about 5.5:1. In some embodiments, the approximate molar ratio of the ionizable lipids to nucleic acid monomer of about 6:1.

In some embodiments, the lipid nanoparticles have a z-average particle diameter (<d>Z) of about 200 nm or less, for example, less than or equal to about 100 nm, or, for instance, less than or equal to about 75 nm. In at least one embodiment of the present disclosure, the lipid nanoparticles have a z-average particle diameter ranging from about 50 nm to about 100 nm, for example, about 60 nm to about 90 nm, from about 70 nm to about 80, such as about 75 nm.

In certain embodiments, the lipid nanoparticles have an encapsulation efficiency (%EE) of nucleic acid segments of about 80% or higher, such as higher than about 90%, such as ranging from about 95%-100%. As used herein, the term “encapsulation efficiency” refers to the ratio of encapsulated nucleic acid segment in the lipid nanoparticles to total nucleic acid segment content in the lipid nanoparticle composition measured by lysis of the lipid nanoparticles using a detergent, e.g., Triton X-100.

Pharmaceutical compositions of the present disclosure may further comprise at least one pharmaceutically acceptable carrier. As used herein, the term “pharmaceutically acceptable carrier” includes compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutical compositions may be in a form suitable for parenteral administration. The pharmaceutical compositions may be in a form suitable for intratracheal instillation, bronchial instillation, and/or inhalation. Pharmaceutical liquid compositions can be nebulized by use of inert gases. Nebulized suspensions may be breathed directly from the nebulizing device or the nebulizing device can be attached to face masks tent, or intermittent positive pressure breathing machine.

The amount of nucleic acid segment that is combined with one or more pharmaceutically acceptable carriers to produce a single dosage form will necessarily vary depending upon the subject treated and the particular route of administration. For further information on routes of administration and dosage regimes the reader is referred to Chapter 25.3 in Volume 5 of Comprehensive Medicinal Chemistry (Corwin Hansch; Chairman of Editorial Board), Pergamon Press 1990.

In one embodiment, the present disclosure provides a method for administering pharmaceutical compositions comprising a plurality of lipid nanoparticles prepared by the present device and/or method, and a therapeutically effective amount of a nucleic acid segment in a subject in need thereof.

The term “subject” includes warm-blooded mammals, for example, primates, cows, pigs, sheep, dogs, cats, rabbits, rats, and mice. In some embodiments, the subject is a primate, for example, a human. In some embodiments, the subject is in need of treatment (e.g., the subject would benefit biologically or medically from treatment).

The lipid nanoparticles prepared by the present device and/or method may further serve as platforms for selective delivery of nucleic acid segments to target cells and tissues, such as antisense oligonucleotides, DNA, mRNAs, siRNAs, Cas9-guideRNA complex. Thus, in one embodiment, is a method of delivering a nucleic acid segment to a cell comprising contacting the cell, in vitro or in vivo, with a pharmaceutical composition comprising a plurality of lipid nanoparticles and a therapeutically effective amount of a nucleic acid segment. In some embodiments, the nucleic acid segment modulates expression, for example, by increasing or decreasing expression, or by upregulating or downregulating expression of the polypeptide.

Another embodiment provides a method for delivering a therapeutically effective amount of a nucleic acid segment to a subject in need thereof, comprising administering to the subject a pharmaceutical composition comprising a plurality of present lipid nanoparticles and a therapeutically effective amount of a nucleic acid segment.

The pharmaceutical compositions comprising a plurality of lipid nanoparticles prepared by the present device and/or method, and a nucleic acid segment disclosed herein may be used to treat a wide variety of disorders and diseases characterized by under expression of a polypeptide in a subject, overexpression of a polypeptide in a subject, and/or absence/presence of a polypeptide in a subject. Accordingly, disclosed are methods of treating a subject suffering from a disease or disorder comprising administering to the subject a pharmaceutical composition comprising a plurality of lipid nanoparticles prepared by the present device and/or method, and a therapeutically effective amount of a nucleic acid segment.

Further disclosed is the use of a pharmaceutical composition comprising a plurality of lipid nanoparticles prepared by the present device and/or method, and a therapeutically effective amount of a nucleic acid segment, to treat a disease or disorder.

Further disclosed is a pharmaceutical composition for use in the treatment of a disease or disorder, wherein the pharmaceutical composition comprises a plurality of lipid nanoparticles prepared by the present device and/or method, and a therapeutically effective amount of a nucleic acid segment.

Further disclosed are methods for increasing protein expression in cells, comprising administering a pharmaceutical composition comprising a plurality of lipid nanoparticles prepared by the present device and/or method, and a nucleic acid segment to a subject in need thereof. In at least one embodiment, protein expression may be increased by a factor of about 2 up to 24 hours. In another embodiment, protein expression may be increased by a factor of about 3 up to 72 hours.

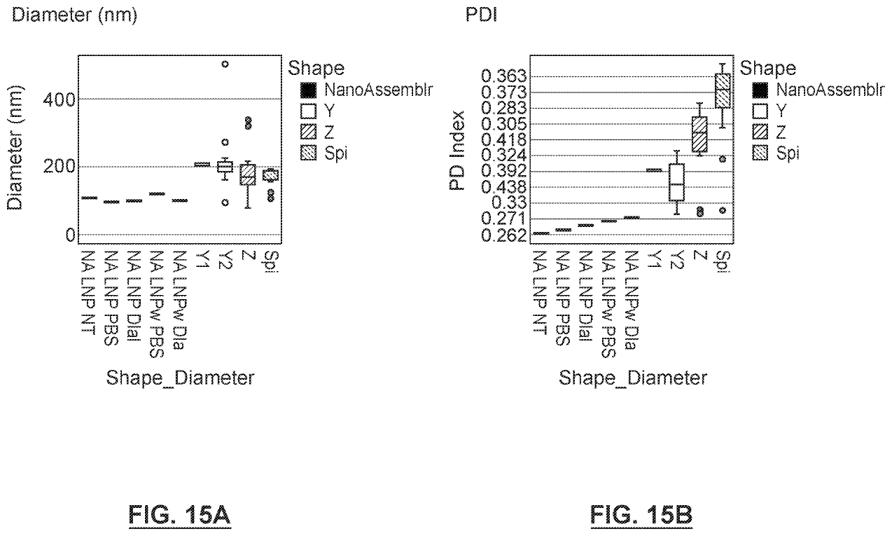

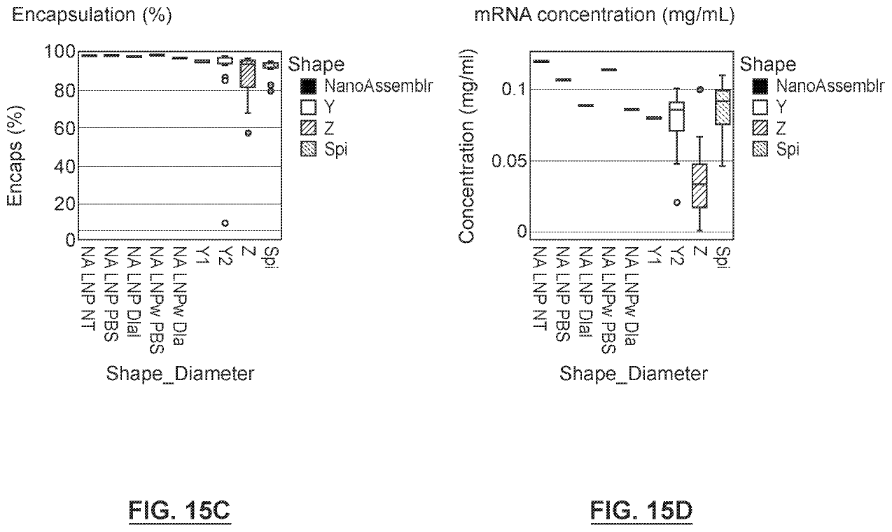

With reference to FIGS. 15A-15D, a comparison of lipid nanoparticles prepared by the devices and/or methods described herein is shown. The lipid nanoparticles referred to within the charts of FIGS. 15A-15D were prepared using MC3 ionizable lipid and polyA as cargo. Specifically, MC3 refers to DLin-MC3-DMA [chemical name: (6Z,9Z,28Z,31Z)-heptatriacont-6,9,28,31-tetraene-19-yl 4-(dimethylamino)butanoate], which is a cationic lipid used to create lipid nanoparticles (LNPs), and polyA refers to mRNAs with polyadenylated tails. Physico-chemical attributes, including diameter size (FIG. 15A), polydispersity index (FIG. 15B), encapsulation (FIG. 15C) and total polyA concentration (FIG. 15D) were measured on all the samples. For each of FIGS. 15A-15D, control or benchmark lipid nanoparticles (LNP) were formulated on the NanoAssemblr® GMP system and processed by standard methods (waste exclusion and dialysis) or equivalently to the other devices for direct comparison (waste inclusion and PBS dilution). More particularly, control or benchmark lipid nanoparticles (LNP) were formulated on the NanoAssemblr® GMP system containing or not the waste fraction (w), post-dialyzed (Dial), diluted (PBS) or not treated (NT).

FIG. 15A is a chart which illustrates the diameter (in nm) of lipid nanoparticles prepared by a first device Y1 including a plurality of fluidic mixing units 104 each having the internal geometry or flowpath 110, a second device Y2 including a plurality of fluidic mixing units 104 each having the internal geometry or flowpath 110, a device Z including a plurality of fluidic mixing units 1004 each having the internal geometry or flowpath 1010, and a device Spi including a plurality of fluidic mixing units 1104 each having the internal geometry or flowpath 1110. The first device Y1 having the internal geometry or flowpath 110 included a cylindrical portion 126 of a constant diameter 400 μm, and the second device Y2 having the internal geometry or flowpath 110 included a cylindrical portion 126 of a constant diameter 600 μm. The device Z included a wavy portion 1059 of a constant diameter 600 μm, and the device Spi included a spiral portion 1159 of a constant diameter 600 μm. As depicted in FIG. 15A, the devices Y1, Y2, Z, and Spi prepared lipid nanoparticles which were comparable in diameter/size to the benchmark lipid nanoparticles prepared by the NanoAssemblr® GMP system.

FIG. 15B is a chart which illustrates the polydispersity (PD) index of lipid nanoparticles prepared by a first device Y1 including a plurality of fluidic mixing units 104 each having the internal geometry or flowpath 110, a second device Y2 including a plurality of fluidic mixing units 104 each having the internal geometry or flowpath 110, a device Z including a plurality of fluidic mixing units 1004 each having the internal geometry or flowpath 1010, and a device Spi including a plurality of fluidic mixing units 1104 each having the internal geometry or flowpath 1110. The first device Y1 having the internal geometry or flowpath 110 included a cylindrical portion 126 of a constant diameter 400 μm, and the second device Y2 having the internal geometry or flowpath 110 included a cylindrical portion 126 of a constant diameter 600 μm. The device Z included a wavy portion 1059 of a constant diameter 600 μm, and the device Spi included a spiral portion 1159 of a constant diameter 600 μm. As depicted in FIG. 15B, the devices Y1, Y2, Z, and Spi prepared lipid nanoparticles having PD Indexes which were comparable to the PD Indexes of the benchmark lipid nanoparticles prepared by a NanoAssemblr® GMP system.

FIG. 15C is a chart which illustrates the encapsulation percentage of lipid nanoparticles prepared by a first device Y1 including a plurality of fluidic mixing units 104 each having the internal geometry or flowpath 110, a second device Y2 including a plurality of fluidic mixing units 104 each having the internal geometry or flowpath 110, a device Z including a plurality of fluidic mixing units 1004 each having the internal geometry or flowpath 1010, and a device Spi including a plurality of fluidic mixing units 1104 each having the internal geometry or flowpath 1110. The first device Y1 having the internal geometry or flowpath 110 included a cylindrical portion 126 of a constant diameter 400 μm, and the second device Y2 having the internal geometry or flowpath 110 included a cylindrical portion 126 of a constant diameter 600 μm. The device Z included a wavy portion 1059 of a constant diameter 600 μm, and the device Spi included a spiral portion 1159 of a constant diameter 600 μm. As depicted in FIG. 15C, the devices Y1, Y2, Z, and Spi prepared lipid nanoparticles having encapsulation percentages which were comparable to the encapsulation percentages of the benchmark lipid nanoparticles prepared by a NanoAssemblr® GMP system.

FIG. 15D is a chart which illustrates the total poly A concentration of lipid nanoparticles prepared by a first device Y1 including a plurality of fluidic mixing units 104 each having the internal geometry or flowpath 110, a second device Y2 including a plurality of fluidic mixing units 104 each having the internal geometry or flowpath 110, a device Z including a plurality of fluidic mixing units 1004 each having the internal geometry or flowpath 1010, and a device Spi including a plurality of fluidic mixing units 1104 each having the internal geometry or flowpath 1110. The first device Y1 having the internal geometry or flowpath 110 included a cylindrical portion 126 of a constant diameter 400 μm, and the second device Y2 having the internal geometry or flowpath 110 included a cylindrical portion 126 of a constant diameter 600 μm. The device Z included a wavy portion 1059 of a constant diameter 600 μm, and the device Spi included a spiral portion 1159 of a constant diameter 600 μm. As depicted in FIG. 15D, the devices Y1, Y2, Z, and Spi prepared lipid nanoparticles having total poly A concentrations which were comparable to the total poly A concentrations of the benchmark lipid nanoparticles prepared by a NanoAssemblr® GMP system.

However, although embodiments of this disclosure are described in the context of lipid nanoparticle production, embodiments of this disclosure may also be used to produce other materials where it is deemed useful. Any system that depends on rapid phase separation to avoid precipitation may benefit from micro-mixing and the combinatorial approach facilitated by embodiments of this disclosure. While embodiments of this disclosure describe mixing lipophilic and hydrophilic components, aqueous components can also be mixed, such as the components for polymeric delivery systems and mRNA, both of which can be soluble in water. Lipid components can also be mixed with complex organic mixtures, such as cell extracts. Pre-formed lipid nanoparticles in aqueous solutions can also be (re)mixed with other components to create particles with new hybrid properties.

While various embodiments according to the present disclosure have been described above, it should be understood that they have been presented by way of illustration and example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A device for producing nanoparticles, the device comprising:
a microplate having a plurality of fluidic mixing units arranged in an array, each fluidic mixing unit defining a flowpath with each flowpath having
a plurality of fluid inlet channels with each fluid inlet channel including an inlet such that, collectively, the inlets of the plurality of fluid inlet channels define a plurality of inlets of the fluidic mixing unit, and
a mixing channel including an outlet that defines a single outlet of the fluidic mixing unit, wherein, along the flowpath, the plurality of fluid inlet channels converge downstream into the mixing channel, and
wherein the array of the plurality of fluidic mixing units includes at least a first set of fluidic mixing units having a first configuration of the flowpath, and a second set of fluidic mixing units having a second configuration of the flowpath, wherein the first configuration is different from the second configuration, and
wherein each fluidic mixing unit of the plurality of fluidic mixing units is configured to produce nanoparticles.

2. The device of claim 1, wherein the microplate includes between 50 and 100 fluidic mixing units.

3. The device of claim 1, wherein the plurality of fluid inlet channels of each fluidic mixing unit includes at least three fluid inlet channels.

4. The device of claim 1, wherein, during formation of the microplate by 3D printing, the plurality of fluidic mixing units are integrally formed with the microplate.

5. The device of claim 1, wherein each fluidic mixing unit of the plurality of fluidic mixing units includes a first end portion, a second end portion opposing the first end portion and including the single outlet of the fluidic mixing unit, and a waist portion extending between the first end portion and the second end portion, the waist portion having a reduced perimeter relative to the first end portion and the second end portion.

6. The device of claim 1, wherein the plurality of fluidic mixing units further includes a third set of fluidic mixing units having a third configuration of the flowpath, the third configuration being different from each of the first configuration and the second configuration.

7. The device of claim 1, wherein the microplate includes a pattern of inlets formed by the plurality of inlets of each fluidic mixing unit and the pattern of inlets is configured to be compatible with a pipetting robot having a plurality of pipettes.

8. The device of claim 7, wherein the pattern of inlets is a two-dimensional rectangular grid with predetermined spacing between adjacent inlets.

9. The device of claim 8, wherein the spacing between adjacent inlets is configured to correspond to a spacing of the plurality of pipettes of the pipetting robot.

10. A system comprising the device of claim 1 and a pipetting robot having a plurality of pipettes, wherein the plurality of inlets of each fluidic mixing unit are configured to receive fluid from a pipette of the plurality of pipettes.

11. The system of claim 10, wherein the inlets of the microplate are configured to receive and mate with tips of the plurality of pipettes.

12. A computer-implemented method of manufacturing a device, the method being implemented by at least one processor executing software instructions, the device including a microplate having a plurality of fluidic mixing units arranged in an array, each fluidic mixing unit defining a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit, wherein, along the flowpath, the plurality of fluid inlet channels converge downstream into the mixing channel, and wherein each fluidic mixing unit of the plurality of fluidic mixing units is configured to produce nanoparticles, the method comprising:
creating, by the at least one processor, a first fluidic mixing unit object having a first configuration of the flowpath;
creating, by the at least one processor, a second fluidic mixing unit object having a second configuration of the flowpath, the first configuration being different from the second configuration;
assembling, by the at least one processor, at least a first set of a plurality of the first fluidic mixing unit objects and a second set of a plurality of the second fluidic mixing unit objects into an array object; and
3D printing the device using the array object.

13. The computer-implemented method of claim 12, wherein the step of 3D printing the device includes generating, by the at least one processor, a printing file from the array object and 3D printing the device with the printing file, the printing file having a file format suitable for 3D printing.

14. The computer-implemented method of claim 13, wherein the file format of the printing file is STL.

15. The computer-implemented method of claim 12, wherein the microplate includes between 50 and 100 fluidic mixing units.

16. The computer-implemented method of claim 12, wherein each fluidic mixing unit of the plurality of fluidic mixing units includes a first end portion, a second end portion opposing the first end portion and including the single outlet of the fluidic mixing unit, and a waist portion extending between the first end portion and the second end portion, the waist portion having a reduced perimeter relative to the first end portion and the second end portion.

17. The computer-implemented method of claim 12, further comprising the step of creating, by the at least one processor, a third fluidic mixing unit object having a third configuration of the flowpath, the third configuration being different from each of the first configuration and the second configuration.

18. The computer-implemented method of claim 17, wherein the step of assembling includes assembling, by the at least one processor, the first set of the plurality of the first fluidic mixing unit objects, the second set of the plurality of the second fluidic mixing unit objects, and a third set of a plurality of the third fluidic mixing unit objects into the array object.

19. The computer-implemented method of claim 12, wherein the microplate includes a pattern of inlets formed by the plurality of inlets of each fluidic mixing unit and the pattern of inlets is configured to be compatible with a pipetting robot having a plurality of pipettes.

20. The computer-implemented method of claim 19, wherein the pattern of inlets is a two-dimensional rectangular grid with predetermined spacing between adjacent inlets.

21. The computer-implemented method of claim 20, wherein the spacing between adjacent inlets is configured to mate with a spacing of the plurality of pipettes of the pipetting robot.

22. A method for producing lipid nanoparticles with a device, the device including a microplate having a plurality of fluidic mixing units arranged in an array, each fluidic mixing unit defining a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit, wherein, along the flowpath, the plurality of fluid inlet channels converge downstream into the mixing channel, and wherein each fluidic mixing unit of the plurality of fluidic mixing units is configured to produce nanoparticles, the method comprising:
injecting a lipid solution into a first inlet of the plurality of inlets of each fluidic mixing unit;
injecting an mRNA solution into a second inlet and a third inlet of the plurality of inlets of each fluidic mixing unit; and
mixing the lipid solution and the mRNA solution within the mixing channel of each fluidic mixing unit for a time sufficient to form lipid nanoparticles, wherein after mixing, the lipid nanoparticles formed exits the single outlet of each fluidic mixing unit.

23. The method of claim 22, wherein the plurality of fluidic mixing units includes at least a first set of fluidic mixing units having a first configuration of the flowpath and a second set of fluidic mixing units having a second configuration of the flowpath, the first configuration being different from the second configuration.

24. The method of claim 22, wherein the step of injecting the lipid solution into the first inlet of each fluidic mixing unit includes injecting a first lipid solution into the first inlet of a first set of fluidic mixing units and injecting a second lipid solution into the first inlet of a second set of fluidic mixing units, the first lipid solution being different from the second lipid solution.

25. The method of claim 22, wherein the step of injecting the mRNA solution into the second inlet and the third inlet of each fluidic mixing unit includes injecting a first mRNA solution into the second inlet and the third inlet of a first set of fluidic mixing units and injecting a second mRNA solution into the second inlet and the third inlet of a second set of fluidic mixing units, the first mRNA solution being different from the second mRNA solution.

26. The method of claim 22, wherein the lipid solution includes at least one lipid dissolved in a lower alcohol or an organic solvent.

27. The method of claim 26, wherein the at least one lipid includes an ionizable lipid, a cholesterol, a phospholipid, and a PEG lipid.

28. The method of claim 27, wherein the at least one lipid is dissolved in ethanol.

29. The method of claim 22, wherein the mRNA solution includes mRNA dissolved in an aqueous buffer solution.

30. The method of claim 22, wherein the microplate includes between 50 and 100 fluidic mixing units.

31. The method of claim 22, wherein each fluidic mixing unit of the plurality of fluidic mixing units includes a first end portion, a second end portion opposing the first end portion and including the single outlet of the fluidic mixing unit, and a waist portion extending between the first end portion and the second end portion, the waist portion having a reduced perimeter relative to the first end portion and the second end portion.

32. The method of claim 22, wherein the steps of injecting the lipid solution and injecting the mRNA solution are performed simultaneously.

33. The method of claim 32, wherein the steps of injecting the lipid solution and injecting the mRNA solution are performed via a pipetting robot having a plurality of pipettes.

34. The method of claim 33, wherein the microplate includes a pattern of inlets formed by the plurality of inlets of each fluidic mixing unit and the pattern of inlets is configured to be compatible with the pipetting robot.

35. The method of claim 34, wherein the pattern of inlets is a two-dimensional rectangular grid with predetermined spacing between adjacent inlets.

36. The method of claim 35, wherein the spacing between adjacent inlets is configured to mate with a spacing of the plurality of pipettes of the pipetting robot.

37. A method of evaluating lipid nanoparticle production with a device, the device including a microplate having a plurality of fluidic mixing units arranged in an array, each fluidic mixing unit defining a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit, wherein, along the flowpath, the plurality of fluid inlet channels converge downstream into the mixing channel, and wherein the array of the plurality of fluidic mixing units includes at least a first set of fluidic mixing units having a first configuration of the flowpath, and a second set of fluidic mixing units having a second configuration of the flowpath, wherein the first configuration is different from the second configuration, and wherein each fluidic mixing unit of the plurality of fluidic mixing units is configured to produce nanoparticles, the method comprising:
injecting a first lipid solution into a first inlet and mRNA into a second inlet and a third inlet of each fluidic mixing unit of the first set of fluidic mixing units;
injecting a second lipid solution into a first inlet and mRNA into a second inlet and a third inlet of each fluidic mixing unit of the second set of fluidic mixing units;
mixing the first lipid solution and mRNA within the mixing channel of each fluidic mixing unit of the first set of fluidic mixing units for a time sufficient to form lipid nanoparticles, wherein after mixing, the lipid nanoparticles formed exits the device through the single outlet of each fluidic mixing unit;
mixing the second lipid solution and mRNA within the mixing channel of each fluidic mixing unit of the second set of fluidic mixing units for a time sufficient to form lipid nanoparticles, wherein after mixing, the lipid nanoparticles formed exits the device through the single outlet of each fluidic mixing unit;
evaluating nanoparticles formed from the first set of fluidic mixing units; and
evaluating nanoparticles formed from the second set of fluidic mixing units.

38. The method of claim 37, wherein the first lipid solution is the same as the second lipid solution.

39. The method of claim 37, wherein the first lipid solution is different from the second lipid solution.

40. The method of claim 37, wherein the mRNA is dissolved in an aqueous buffer solution.

41. The method of claim 37, wherein each of the first lipid solution and the second lipid solution includes at least one lipid dissolved in a lower alcohol or an organic solvent.

42. The method of claim 41, wherein the at least one lipid is selected from the group consisting of an ionizable lipid, a cholesterol, a phospholipid, and a PEG lipid.

43. The method of claim 42, wherein the at least one lipid includes an ionizable lipid, a cholesterol, a phospholipid, and a PEG lipid.

44. The method of claim 42, wherein the at least one lipid is dissolved in ethanol.

45. The method of claim 37, wherein the microplate includes between 50 and 100 fluidic mixing units.

46. The method of claim 37, wherein each fluidic mixing unit of the plurality of fluidic mixing units includes a first end portion, a second end portion opposing the first end portion and including the single outlet of the fluidic mixing unit, and a waist portion extending between the first end portion and the second end portion, the waist portion having a reduced perimeter relative to the first end portion and the second end portion.

47. The method of claim 37, wherein the steps of injecting are performed simultaneously.

48. The method of claim 47, wherein the steps of injecting are performed via a pipetting robot having a plurality of pipettes.

49. The method of claim 48, wherein the microplate includes a pattern of inlets formed by the plurality of inlets of each fluidic mixing unit and the pattern of inlets is configured to be compatible with the pipetting robot.

50. The method of claim 49, wherein the pattern of inlets is a two-dimensional rectangular grid with predetermined spacing between adjacent inlets.

51. The method of claim 50, wherein the spacing between adjacent inlets is configured to mate with a spacing of the plurality of pipettes of the pipetting robot.

52. The method of claim 37, further comprising comparing the nanoparticles formed from the first configuration with the nanoparticles formed from the second configuration.

53. The method of claim 37, wherein the step of evaluating the nanoparticles formed from the first set of fluidic mixing units and the step of evaluating the nanoparticles formed from the second set of fluidic mixing units include evaluating at least one parameter of the nanoparticles, the parameter being selected from the group consisting of a size of the nanoparticles, a surface charge of the nanoparticles and an encapsulation efficiency of the nanoparticles.

54. A method of evaluating lipid nanoparticle production with a device, the device including a microplate having a plurality of fluidic mixing units arranged in an array, each fluidic mixing unit defining a flowpath with each flowpath having a plurality of fluid inlet channels with each fluid inlet channel including an inlet such that, collectively, the inlets of the plurality of fluid inlet channels define a plurality of inlets of the fluidic mixing unit, and a mixing channel including an outlet that defines a single outlet of the fluidic mixing unit, wherein, along the flowpath, the plurality of fluid inlet channels converge downstream into the mixing channel, and wherein the array of the plurality of fluidic mixing units includes at least a first set of fluidic mixing units and a second set of fluidic mixing units, and wherein each fluidic mixing unit of the plurality of fluidic mixing units is configured to produce nanoparticles, the method comprising:
injecting a first lipid solution into a first inlet and mRNA into a second inlet and a third inlet of each fluidic mixing unit of the first set of fluidic mixing units;
injecting a second lipid solution into a first inlet and mRNA into a second inlet and a third inlet of each fluidic mixing unit of the second set of fluidic mixing units, wherein the first lipid solution is different from the second lipid solution;
mixing the first lipid solution and the mRNA within the mixing channel of each fluidic mixing unit of the first set of fluidic mixing units for a time sufficient to form lipid nanoparticles, wherein after mixing, the lipid nanoparticles formed exits the device through the single outlet of each fluidic mixing unit;
mixing the second lipid solution and the mRNA within the mixing channel of each fluidic mixing unit of the second set of fluidic mixing units for a time sufficient to form lipid nanoparticles, wherein after mixing, the lipid nanoparticles formed exits the device through the single outlet of each fluidic mixing unit;
evaluating nanoparticles formed from the first set of fluidic mixing units; and
evaluating nanoparticles formed from the second set of fluidic mixing units.

55. The method of claim 54, wherein the first set of fluidic mixing units has a first configuration of the flowpath, and the second set of fluidic mixing units has a second configuration of the flowpath, wherein the first configuration is different from the second configuration.

56. The method of claim 54, wherein the first set of fluidic mixing units has a first configuration of the flowpath, and the second set of fluidic mixing units has a second configuration of the flowpath, wherein the first configuration is the same as the second configuration.

57. The method of claim 54, wherein the mRNA is dissolved in an aqueous buffer solution.

58. The method of claim 54, wherein each of the first lipid solution and the second lipid solution includes at least one lipid dissolved in a lower alcohol or an organic solvent.

59. The method of claim 58, wherein the at least one lipid is selected from the group consisting of an ionizable lipid, a cholesterol, a phospholipid, and a PEG lipid.

60. The method of claim 58, wherein the at least one lipid includes an ionizable lipid, a cholesterol, a phospholipid, and a PEG lipid.

61. The method of claim 58, wherein the at least one lipid is dissolved in ethanol.

62. The method of claim 54, wherein the microplate includes between 50 and 100 fluidic mixing units.

63. The method of claim 54, wherein each fluidic mixing unit of the plurality of fluidic mixing units includes a first end portion, a second end portion opposing the first end portion and including the single outlet of the fluidic mixing unit, and a waist portion extending between the first end portion and the second end portion, the waist portion having a reduced perimeter relative to the first end portion and the second end portion.

64. The method of claim 54, wherein the steps of injecting are performed simultaneously.

65. The method of claim 54, wherein the steps of injecting are performed via a pipetting robot having a plurality of pipettes.

66. The method of claim 65, wherein the microplate includes a pattern of inlets formed by the plurality of inlets of each fluidic mixing unit and the pattern of inlets is configured to be compatible with the pipetting robot.

67. The method of claim 66, wherein the pattern of inlets is a two-dimensional rectangular grid with predetermined spacing between adjacent inlets.

68. The method of claim 67, wherein the predetermined spacing between adjacent inlets is configured to mate with a spacing of the plurality of pipettes of the pipetting robot.

69. The method of claim 54, further comprising comparing the nanoparticles formed from the first configuration with the nanoparticles formed from the second configuration.

70. The method of claim 54, wherein the step of evaluating the nanoparticles formed from the first set of fluidic mixing units and the step of evaluating the nanoparticles formed from the second set of fluidic mixing units include evaluating at least one parameter of the nanoparticles, the parameter being selected from the group consisting of a size of the nanoparticles, a surface charge of the nanoparticles and an encapsulation efficiency of the nanoparticles.
